(19) United States
(12) Patent Application Publication
(10) Pub. No.: US 2018/0284141 A1
(43) Pub. Date: Oct. 4, 2018

(54) METHOD FOR PREDICTING RISK OF COGNITIVE DETERIORATION

(71) Applicant: CRC for Mental Health Ltd, Carlton South, Victoria (AU)

(72) Inventors: Scott Ayton, Caulfield (AU); Noel Faux, Carlton South (AU); Ashley Ian Bush, Melbourne (AU)

(73) Assignee: CRC For Mental Health Ltd, Carlton South, Victoria (AU)

(21) Appl. No.: 15/562,801

(22) PCT Filed: Apr. 1, 2016
(86) PCT No.: PCT/AU2016/050248

§ 371 Date: Sep. 28, 2017

(30) Foreign Application Priority Data
Apr. 2, 2015 (AU) 2015901210
Feb. 3, 2016 (AU) 2016900347

(51) Int. Cl. G01N 33/84; G01N 33/68; A61K 31/4412; A61P 25/28; C12Q 1/6883; A61B 5/055; A61B 5/00

(52) U.S. Cl. CPC G01N 33/84; G01N 33/6896; A61K 31/4412; A61P 25/28; C12Q 1/6883; G01N 2800/50; A61B 5/4088; A61B 5/7275; G01N 2800/2821; G01N 2333/775; G01N 2800/2814; A61B 5/055

(57) ABSTRACT

The present invention relates to methods for predicting a risk of cognitive deterioration, monitoring progression of cognitive deterioration and diagnosing cognitive deterioration in a patient. The present invention further relates to methods for diminishing progression rate of cognitive deterioration in a patient by lowering brain iron levels in the patient or lowering CSF ferritin levels in the patient.

### FIELD OF THE INVENTION

The present invention relates to methods for predicting risk of cognitive deterioration relating to the areas of dementias, cognitive disorders and/or affective disorders and/or behavioural dysfunction, Alzheimer's Disease and related dementias. More particularly, it relates to genetic vulnerability, prognostic methods and treatment methods. It relates to a correlation between brain iron and cognitive deterioration. Preferably the invention relates to ferritin or more preferably cerebrospinal fluid (CSF) ferritin as an indicator of the brain iron levels in methods, for the diagnosis, prognosis and/or monitoring progression of cognitive deterioration and stratifying an individual into one or more classes depending on the diagnosis or prognosis of the cognitive deterioration. More specifically, the present invention relates to the diagnosis, prognosis and monitoring of Alzheimer's disease (AD) in a subject or stratifying individuals with the disorder by a determination of brain iron levels correlating with genotype as an AD biomarker.

### BACKGROUND

The already extensive burden of Alzheimer's disease (AD) to Australia is projected to increase due to an aging population demographic and no effective treatments. Recent large-scale phase III clinical trials of drugs targeting known pathways involved in AD have failed to benefit patients. There is an emerging consensus that disease-modifying treatments should be delivered during the pre-clinical phase of the disease, as amyloid β (Aβ) pathology begins to accumulate. Early detection of AD is therefore necessary for effectively treating this disease. There is currently no clinically acceptable prognostic biomarker for AD and the associated conditions leading to AD such as cognitive deterioration.

AD brain pathology starts developing approximately two decades prior to the onset of cognitive symptoms. Consequently, anti-AD therapies may have the best chance of success when given in this preclinical period. There is a need to identify biomarkers that predict cognitive deterioration early in AD. Amyloid PET imaging is the most advanced biomarker of geriatric cognitive deterioration. High Aβ burden (Aβ+), identified by PiB, flutemetamol, or florbetapir radioligands, predicts cognitive decline with an average effect size (difference between slopes) of ˜0.5 on memory composite scores in cognitively normal (CN) subjects over 3+ years. Aβ imaging is a sensitive predictor (98%) of cognitive decline but studies have shown repeatedly a large prevalence (˜20-30%) of cognitive unimpaired people over age 60 with already high Aβ burden in the brain. It is now clear that other factors are necessary to precipitate cognitive decline toward Alzheimer's dementia.

Post-mortem studies have shown that tau deposition correlates more strongly than Aβ burden with cognitive impairment. Attempts have been made to diagnose or differentially diagnose AD by measuring the level of a target such as tau and Aβ in the patient whose level specifically increases or decreases in the cerebrospinal fluid (“CSF”) of a dementia patient.

Aβ and tau form the brain amyloid and tangle proteopathies of AD and have been the subjects of extensive biomarker research. The accumulation of cortical amyloid and hippocampal tau are pathognomonic of AD, but can also be substantial in people regarded as clinically normal.

It is now understood that, on its own, the prognostic and diagnostic value of Aβ is limited, whether this is measured in biofluids or via Positron Emission Tomography (PET) imaging. Post-mortem studies find brain tau accumulation in normal ageing, and while elevated CSF tau is one of the best available prognostic biomarkers, it is not yet clinically useful.

In light of the above, there is a need for an improved method of identifying those with cognitive deterioration leading to neurological disorders such as AD or those displaying cognitive decline, particularly at the onset of the disease, which may assist in delaying disease progression. The ability to detect preclinical or early stage disease through reliable measurement of markers present in biological samples from a subject suspected of having AD would also allow treatment and management of the disease to begin earlier. The same tests can be used to monitor the progression of decline without the need for expensive equipment, discomfort and side effects experienced in the present available methods of diagnosis and prognosis.

A test which can provide assistance to clinicians in reaching an early stage prognosis prior to the portrayal of detectable clinical indicators and which would obviate the need for actual confirmatory brain imaging tests would be useful.

With disease modifying therapies for AD undergoing clinical trials, there is a social and economic imperative to identify biomarkers that can detect features of the disease in at-risk individuals in the earliest possible stage, so anti-AD therapy can be administered at a time when the disease burden is mild and it may prevent or delay functional and irreversible cognitive loss.

Accordingly, there is a desire to provide a simple and effective measure of cognitive deterioration in patients that can be used to diagnose, prognose or monitor a patient with a cognitive deterioration and that correlates with the cognitive deterioration in the patient. This early detection may assist in delaying the onset of AD if treated early and appropriately or to monitor progression of a patient undergoing drug therapy for cognitive deterioration.

### SUMMARY OF THE INVENTION

Measuring cognitive deterioration before the onset of AD may enable early treatment with drugs that would delay disease progression.

Accordingly, in an aspect of the present invention there is provided a method for predicting a risk of cognitive deterioration in a patient, said method comprising:
determining a first level of brain iron in a patient;
comparing the first level of brain iron to a reference level of brain iron;
determining a difference between the first level of brain iron and the reference level; and
deducing a risk for cognitive deterioration in the patient from the difference.

Applicants have identified brain iron elevation as an alternative/adjunct prognostic for cognitive deterioration leading to AD. They show that iron burden of the brain has an impact on longitudinal outcomes of AD (cognition, brain atrophy) similar in magnitude to the more established biomarkers of the disease (e.g. CSF tau and Aβ).

In an embodiment of the present invention, the levels of brain iron may be determined as a measure of any iron related protein levels such as but not limited to ceruloplasmin, amyloid precursor protein, tau, ferritin, transferrin, and transferrin binding protein. Preferably, the brain iron is determined by ferritin levels or by MRI or by any method available to the skilled addressee. In a preferred embodiment the level of brain iron is determined as a measure of cerebrospinal fluid (CSF) ferritin.

Using the major iron binding protein ferritin in CSF as an index, high brain-iron load was associated with poorer cognition and brain atrophy over 6-7 years in a cohort of cognitively normal, mild cognitive impairment and AD subjects.

In another aspect of the invention there is provided a method of diagnosing cognitive deterioration in a patient said method comprising:
determining a first level of brain iron in a patient;
comparing the first level of brain iron to a reference level of brain iron;
determining a difference between the first level of brain iron and the reference level;
deducing cognitive deterioration in the patient from the difference.

In yet another aspect of the present invention there is provided a method for monitoring progression of cognitive deterioration in a patient, said method comprising:
determining a level of brain iron in the patient at first time point;
determining a level of brain iron at in the same patient at a second time point which is after the first time point;
optionally comparing the levels of brain iron from the first and second time points to a reference level;
determining a difference in the levels of brain iron at each of the first and second time points;
deducing progression of cognitive deterioration from the difference in brain iron levels from the first and the second time points.

The changes in the levels of brain iron can additionally be used in assessing for any changes in cognitive deterioration of a patient. Accordingly, in the monitoring of the levels of brain iron, it is possible to monitor for the presence of cognitive deterioration over a period of time, or to track cognitive deterioration progression in a patient.

In another embodiment of the invention the method for determining cognitive deterioration further includes:
determining an apolipoprotein E (ApoE) level in the patient;
comparing the level of Apo E in the patient to a reference level of Apo E;
determining a correlation between the Apo E level in the patient and the reference level to the brain iron levels corresponding to the patient and the reference level of brain iron; and
deducing a risk of cognitive deterioration from the correlation between the Apo E levels and the brain iron levels.

Applicants have found that CSF ferritin levels formed a remarkable association with CSF ApoE levels and subjects with APOE ε4 isoform have elevated CSF ferritin compared to subjects without the AD risk allele.

In yet another embodiment, the present method further includes determining a level of a biomarker of cognitive impairment such as but not limited to Tau or Aβ used singularly or in combination with the method to assess cognitive deterioration. These additional markers may enhance the accuracy of the method for determining a risk of cognitive deterioration.

In another aspect of the invention there is provided a method for diminishing progression rate of cognitive deterioration, said method comprising lowering brain iron levels.

In another aspect of the invention there is provided a method for diminishing progression rate of cognitive deterioration, said method comprising lowering CSF ferritin levels.

In yet another aspect of the invention there is provided a method for increasing cognitive performance, said method comprising lowering CSF ferritin levels.

To lower brain iron or CSF ferritin levels compounds such as iron chelators such as Deferiprone may be used.

### BRIEF DESCRIPTION OF THE FIGURES

FIG. 1 shows conversion from MCI to dementia as predicted by baseline CSF biomarkers. Based on the minimal Cox proportional hazards model (cf. Table 4), the conversion is plotted for each quintile of (a) ApoE (ferritin=6·5 ng/mL, tau/Aβ1-42=0·69 units) and (b) tau/Aβ1-42 (ferritin=6·5 ng/mL, ApoE=7·2 μg/mL). The numbers on the right side of the graphs indicate the quintile boundaries.

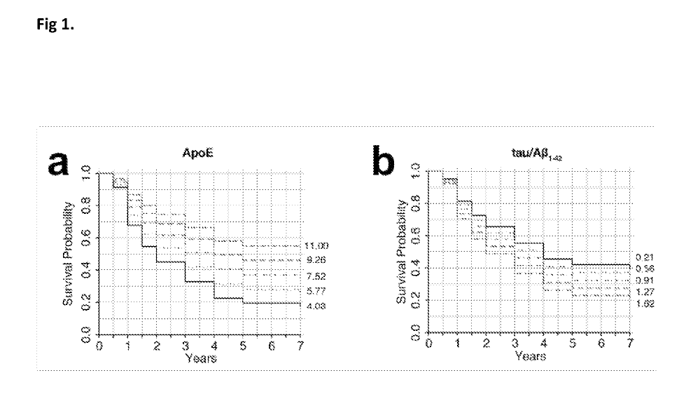

FIG. 2 shows utility of CSF ferritin as a biomarker for MCI conversion to AD. Receiver operating curves of logistic regression modelling of MCI conversion to AD (cf. Table 4). (a) Base model containing the demographic information: age, gender, BMI, years of education, and APOE ε4 status. (b) Base model plus CSF ferritin. (c) Base model plus CSF ApoE. (d) Base model plus tau/Aβ₁₋₄₂. AUC—Area Under Curve.

FIG. 3 shows CSF ferritin associates with ApoE levels and varies according to APOE genotype. (a,b) Modelling ferritin in CSF. (M3 of Supplementary Table 1). Minimal multiple regression contained CSF ApoE and APOE ε4. (a) Scatterplot of CSF ApoE and ferritin levels in APOE ε4 carriers and non-ε4 carriers. The genotype did not affect the relationship between ApoE and ferritin; however, genotype is associated with CSF ferritin levels, and thus ε4 carriers and non-ε4 carriers are plotted separately. The R2 for the linear component of the full model was 0.341 (displayed on graph). (b) CSF Ferritin levels in APOE ε4 carriers and noncarriers (ANCOVA: P-value=1.10×10⁻⁸). (c) Multiple regression of CSF ApoE. ApoE levels in APOE ε4 carriers and non-carriers (ANCOVA: P=2.50×10⁻⁰⁹). Data are means±s.e. ‘n’ is represented in graph columns.

FIG. 4 shows CSF ferritin levels independently predict cognitive status. (a-c) Multiple regression of baseline ADAS-Cog13 score expressed as tertiles of CSF (a) ferritin (L<5.5; H>7.3 ng m⁻¹), (b) ApoE (L<5.8; H>7.8 mg ml⁻¹) and (c) tau/Ab₁₋₄₂ (L<0.35; H>0.76). (d) Multiple regression of baseline RVLT score expressed as CSF ferritin tertiles. Data are adjusted for baseline diagnosis, gender, years of education and the AD CSF biomarkers in the minimal models. Data are means±s.e. ‘n’ is shown in graph columns. CN, cognitively normal; MCI, mild cognitive impairment.

FIG. 5 shows conversion from MCI to dementia as predicted by baseline CSF biomarkers. (a) MCI survival based on the minimal Cox proportional hazards model (Table 2), the conversion is plotted for each quintile of ferritin (applying mean values for the cohort: ApoE=7.2 mg ml⁻¹, tau/Ab₁₋₄₂=0.69 units). The numbers on the right side of the graphs indicate the quintile boundaries. This minimal model contained only the CSF biomarkers. (b) Change in mean age of diagnosis according to CSF biomarkers. The months taken for B50% survival of each quintile boundary in the Cox models were graphed against the unit values of those boundaries. The gradient of the linear model was used to estimate change in age of conversion for each unit change in analyte (compare with FIG. 5a and FIG. 1). (c-e) Receiver operating curves of logistic regression modelling of MCI conversion to AD (Table 2,). (c) Base model controlling for age, gender, BMI, years of education and APOE ε4 status. (d) Base model plus ApoE and tau/Ab₁₋₄₂. (e) Base model plus ApoE, tau/Ab₁₋₄₂ and ferritin. AUC, area under curve.

FIG. 6 shows CSF ferritin levels independently predict brain structural changes. (a-c) Longitudinal hippocampal changes based on tertiles of CSF (a) ferritin (L<5.5; H>7.3 ng ml⁻¹) (b) ApoE (L<5.8; H>7.8 mg ml⁻¹) and (c) tau/Ab₁₋₄₂ (L<0.35; H>0.76) tertiles. (d-f) Longitudinal lateral ventricular changes based on CSF (d) ferritin (e) ApoE and (f) tau/Ab₁₋₄₂ tertiles. These mixed effects models were adjusted for age, gender, baseline diagnosis, years of education, APOE ε4 status and intracranial volume. Tertiles at baseline were not significantly different for all models, therefore for visual display the baseline values were held at the adjusted means for each diagnostic group. CN, cognitively normal; H, highest tertile; M, middle tertile; MCI, mild cognitive impairment; L, lowest tertile.

FIG. 7 shows a schematic for the impact of ferritin and other biomarkers on AD presentation. (a) CSF ferritin has a qualitatively different impact to (b) CSF tau/Ab₁₋₄₂ and ApoE on cognitive performance over time in cognitively normal (dotted lines) and in subjects who develop AD (solid lines). Higher CSF ferritin levels are associated with poorer baseline cognitive status (for example, RVLT) by [α] points, where [α]=Ln[ferritin (ng ml⁻¹)]*1 77 (Table 2). This effect is constant over time, such that [α]=[β,γ]. Consequently, ferritin causes a shift to the left in age of conversion to AD by [δ] months, where [δ]=ferritin (ng ml⁻¹)*3 (FIG. 5b). Levels of tau/Ab₁₋₄₂ or ApoE are associated with both baseline cognitive status [ε] and the rate of cognitive deterioration, such that [ε]<[φ, γ]. The effect causes a shift in age of diagnosis by [η] months where [η]=ApoE (mg ml⁻¹)*8 or tau/Ab₁₋₄₂ (units)*17 (FIG. 5b).

FIG. 8 shows cognitive decline in Cognitively Normal (CN) subjects as predicted by baseline CSF factors stratified by APOE-ε4 allelic status. (A-B) Association between baseline (A) CSF tau/Aβ1-42 ratio, and (B) CSF ferritin, with annual change in RAVLT score in APOE ε4 carriers and non-carriers over 7 years. (C-D) Association between baseline (C) CSF tau/Aβ₁₋₄₂ ratio, and (D) CSF ferritin, with annual change in ADAS-Cog13 score in APOE ε4 carriers and non-carriers over 7 years. (E) ROC of baseline CSF ferritin for predicting stable or deteriorating (≥1 RAVLT unit change per year) cognition in CN ε4 subjects over 7 years. Area under the curve (AUC)=0.96.

### DETAILED DESCRIPTION OF THE INVENTION

Measuring cognitive deterioration before the onset of AD may enable early treatment intervention to delay disease progression. Anti-AD therapies given in the pre-clinical period will have the best chance of success. However, in some cases dementia or

AD may not fully develop, but the patient displays symptoms of Mild Cognitive Impairment (MCI) or are cognitively normal elders who may eventually experience cognitive deterioration. Monitoring progression will be imperative for managing the cognitive deterioration over time.

Accordingly, in an aspect of the present invention there is provided a method for predicting a risk of cognitive deterioration in a patient, said method comprising:
determining a first level of brain iron in a patient;
comparing the first level of brain iron to a reference level of brain iron;
determining a difference between the first level of brain iron and the reference level; and
deducing a risk for cognitive deterioration in the patient from the difference.

Applicants have identified brain iron elevation as an alternative/adjunct prognostic for cognitive deterioration leading to AD. Iron accumulates in affected regions during the disease but, until recently, there was debate about its impact on pathogenesis. They have quantified the contribution of brain iron on progression of AD. Applicants show that iron burden of the brain has an impact on longitudinal outcomes of AD (cognition, brain atrophy) similar in magnitude to the more established biomarkers of the disease (e.g. CSF tau and Aβ). These findings, in combination with growing evidence implicating iron elevation in AD pathogenesis, has provided support for brain iron levels as a biomarker for AD using MRI and advanced techniques.

Iron elevation in AD is an unexplored, putative co-determinate of cognitive decline. Until recently, the contribution of iron to AD pathogenesis was unclear. Here applicants show the impact of iron on longitudinal AD outcomes.

The present invention relates to assessing a risk of cognitive deterioration measured as a degree of decline in cognitive capacity. When a patient's cognitive capacity declines changes occur which give rise to a variety of symptoms associated with aging, such as forgetfulness, decreased ability to maintain focus, and decreased problem solving capability. symptoms oftentimes progress into more serious conditions, such as dementia and depression, or even Alzheimer's disease.

Mild cognitive impairment (MCI) is an intermediate stage between the expected cognitive decline of normal aging and the more serious decline of dementia. It can involve problems with memory, language, thinking and judgment that are greater than normal age-related changes. Mild cognitive impairment causes cognitive changes that are serious enough to be noticed by the individuals experiencing them or to other people, but the changes are not severe enough to interfere with daily life or independent function.

Currently, the clinical diagnosis in the areas of dementias, cognitive disorders and/or affective disorders and/or behavioural dysfunction, Alzheimer's Disease and related dementias generally requires an evaluation of medical history and physical examination including neurological, neuropsychological and psychiatric assessment including memory and/or psychological tests, assessment of language impairment and/or other focal cognitive deficits (such as apraxia, acalculia and left-right disorientation), assessment of impaired judgment and general problem-solving difficulties, assessment of personality changes ranging from progressive passivity to marked agitation, as well as various biological, radiological and electrophysiological tests, such as for instance measuring brain volume or activity measurements derived from neuroimaging modalities such as magnetic resonance imaging (MRI) or positron emission tomography (PET) of relevant brain regions. Applicants have found a correlation between brain iron, ferritin and CSF ferritin and cognitive function that will enable a simple assessment of the risk for cognitive deterioration in these patients.

As used herein, reference to cognitive deterioration includes mild cognitive impairment (MCI), MCI conversion to Alzheimer's Disease (AD), and AD. However, the invention also relates broadly to the areas of dementias, cognitive disorders and/or affective disorders and/or behavioural dysfunction, Alzheimer's Disease and related dementias. The term “cognitive deterioration” may be used interchangeably with “cognitive decline”.

The term “cognitively normal (CN) patient” as used herein means a subject which has no significant cognitive impairment or impaired activities of daily living. Patients that are suspected of, or are at risk of cognitive deterioration may be compared against a CN patient. This includes patients that are cognitively normal but show changed levels of a marker indicative of a neurological disease such as amyloid loading in the brain (preferably determined by PET imaging). The characteristics of a CN patient will assist in providing a reference level or reference value to which deterioration from normal can be determined. Preferably, the CN patient does not carry an Apo ε4 allele.

A risk of cognitive deterioration may be assessed relative to the CN patient which will provide a reference level. Patients who are at risk of cognitive deterioration and/or Alzheimer's Disease include those with family histories, genetic vulnerability and deficiency alleles. They may be vulnerable and carry genes which predispose them to a more rapid cognitive deterioration leading to dementia and AD.

Patients who can be tested and/or treated according to any of the methods of the present invention include those who present with cognitive dysfunction with a history of treated depression, cognitive dysfunction with a history of depression, cognitive dysfunction with bipolar disease or schizoaffective disorders, cognitive dysfunction with generalized anxiety disorder, cognitive dysfunction with attention deficit, ADHD disorder or both attention deficit and ADHD disorder, dyslexia, developmental delay, school adjustment reaction, Alzheimer's Disease, amnesic mild cognitive impairment, non-amnesic mild cognitive impairment, cognitive impairment with white matter disease on neuroimaging or by clinical examination, frontotemporal dementia, cognitive disorders in those under 65 years of age, those with serum homocysteine levels of less than 10 nmol/l, and those with high serum transferrin levels (uppermost population quartile).

As used herein, the terms “individual,” “subject,” and “patient,” generally refer to a human subject, unless indicated otherwise, e.g., in the context of a non-human mammal useful in an in vivo model (e.g., for testing drug toxicity), which generally refers to murines, simians, canines, felines, ungulates and the like (e.g., mice, rats, other rodents, rabbits, dogs, cats, swine, cattle, sheep, horses, primates, etc.).

The terms “determining,” “measuring,” “evaluating,” “assessing,” and “assaying,” as used herein, generally refer to any form of measurement, and include determining if an element is present or not in a biological sample. These terms include both quantitative and/or qualitative determinations, which require sample processing and transformation steps of the biological sample. Assessing may be relative or absolute. The phrase “determining a level of” can include determining the amount of something present, as well as determining whether it is present or absent.

A level of brain iron may be determined from a patient suspected of having cognitive deterioration or the same patient from another time period. Alternatively, a level of brain iron may be determined from a patient that is known not to have cognitive deterioration providing a reference value or reference level or a control level. Preferably this will be from a healthy control or a cognitively normal individual (CN).

As used herein, a “reference value” or “reference level” may be used interchangeably and may be selected from the group comprising an absolute value; a relative value; a value that has an upper and/or lower limit; a range of values; an average value; a median value, a mean value, a shrunken centroid value, or a value as compared to a particular control or baseline value. Preferably it is a predetermined reference value obtained from a known sample prepared in parallel with the biological or test sample in question. It is to be understood that other statistical variables may be used in determining the reference value. A reference value can be based on an individual sample value, such as for example, a value obtained from a sample from the individual with cognitive deterioration, but at an earlier point in time, or a value obtained from a sample from a patient or another patient with the disorder other than the individual being tested, or a “normal” or “healthy” individual, that is an individual not diagnosed with cognitive deterioration otherwise a CN individual. The reference value can be based on a large number of reference samples, such as from AD patients or patients with cognitive deterioration, normal individuals or based on a pool of samples including or excluding the sample to be tested.

For diagnostic and prognostic methods, the “reference level” is typically a predetermined reference level, such as an average of levels obtained from a population that is afflicted with cognitive deterioration. In some instances, the predetermined reference level is derived from (e.g., is the mean or median of) levels obtained from an age-matched population. In some examples disclosed herein, the age-matched population comprises individuals with non-AD or neurodegenerative disease individuals.

For methods providing a prognosis of cognitive deterioration or a risk of cognitive deterioration, a reference level may also be considered as generally a predetermined level considered “normal” for the particular diagnosis (e.g., an average level for age-matched individuals not diagnosed with cognitive deterioration or an average level for age-matched individuals diagnosed with cognitive deterioration other than AD and/or healthy age-matched individuals), although reference levels which are determined contemporaneously (e.g., a reference value that is derived from a pool of samples including the sample being tested) are also contemplated.

A reference level may also be a measure of a constant internal control to standardize the measurements of the first level and reference level to decrease the variability between the tests. The internal control may be a sample from a blood bank such as the Red Cross.

Hence in conducting the method of the present invention, a set of samples can be obtained from individuals having cognitive deterioration and a set of samples can be obtained from individuals not having cognitive deterioration.

The measured level of brain iron may be a primary measurement of the level of bound or unbound iron in the brain or it may be a secondary measurement of the iron (a measurement from which the quantity of the iron can be determined but not necessarily deduced (qualitative data)), such as a measure of iron related protein levels such as ferritin. Hence, a sample may be processed to exclude unbound cellular iron if measuring iron related protein levels like ferritin levels.

In an embodiment of the present invention, the levels of brain iron may be determined as a measure of any iron related protein levels such as but not limited to ceruloplasmin, amyloid precursor protein, tau, ferritin, transferrin, transferrin binding protein etc. Preferably, the brain iron is determined by ferritin levels or by MRI or sonography or by any method available to the skilled addressee.

Accordingly the invention provides a use of iron related protein levels (e.g. ceruloplasmin, amyloid precursor protein, tau, ferritin, transferrin, transferrin binding protein etc.), in conjunction with information regarding APOE genotype, CSF tau, Aβ and ApoE levels, to predict the rate of cognitive decline in normal people and individuals with MCI.

Ferritin is the iron storage protein of the body and is elevated in AD brain tissue. In cultured systems, ferritin expression and secretion by glia is dependent on cellular iron levels. Ferritin levels in CSF likely reflect iron levels in the brain and can have clinical utility.

Accordingly, in a preferred embodiment the level of brain iron is determined as a measure of cerebrospinal fluid (CSF) ferritin. Hence the invention provides use of a measurement of CSF ferritin concentration, (in conjunction with information regarding APOE genotype, CSF tau, Aβ and ApoE levels) to predict the rate of cognitive decline in an individual who preferably exhibits the symptoms of mild cognitive impairment (MCI).

In another embodiment there is provided a use of a measurement of CSF ferritin concentration, (preferably in conjunction with information regarding APOE genotype, CSF tau, Aβ and ApoE levels) to predict the rate of cognitive decline in an individual who exhibits no symptoms (normal).

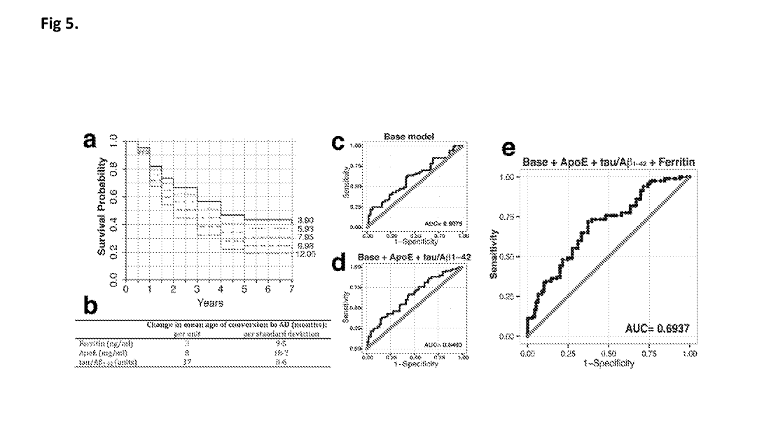

Using the major iron binding protein ferritin in CSF as an index, high brain-iron load was associated with poorer cognition (e.g. ADAS-Cog; FIG. 5a) and brain atrophy (e.g. Lateral ventricle-structural MRI; FIG. 5b) over 6-7 years in a cohort of cognitively normal (n=91), mild cognitive impairment (n=144) and AD (n=67) subjects. The magnitude impact of CSF ferritin on these and other AD-outcomes is comparable to the tau/Aβ42 ratio—the gold standard CSF biomarker for AD. CSF ferritin independently predicted progression to AD over the study period (FIG. 5c) and improved the predictive potential of the tau/Aβ. Each 1 ng/ml increase in ferritin brought forward diagnosis by 3 months. Thus, applicants have demonstrated a role for brain iron in AD, and present brain iron as a target for AD prognosis.

In performing the presently claimed method the level of brain iron, preferably ferritin or more preferably CSF ferritin is preferably identified. As would be appreciated by one of skill in the art, the level (e.g., concentration, expression and/or activity) of brain iron, preferably ferritin or more preferably CSF ferritin can be qualified or quantified. Preferably, the level of brain iron, preferably ferritin or more preferably CSF ferritin is quantified as a level of DNA, RNA, lipid, carbohydrate, protein, metal or protein expression.

It will be apparent that numerous qualitative and quantitative techniques can be used to identify the level of brain iron, preferably ferritin or more preferably CSF ferritin. These techniques may include 2D DGE, mass spectrometry (MS) such as multiple reaction monitoring mass spectrometry (MRM-MS), Real Time (RT)-PCR, nucleic acid array; ELISA, functional assay, by enzyme assay, by various immunological methods, or by biochemical methods such as capillary electrophoresis, high performance liquid chromatography (HPLC), thin layer chromatography (TLC), hyper-diffusion chromatography, two-dimensional liquid phase electrophoresis (2-D-LPE) or by their migration pattern in gel electrophoreses or MRI.

However, it will be apparent to the skilled addressee that the appropriate technique used to identify the level of brain iron, preferably ferritin or more preferably CSF ferritin will depend on the characteristics of the molecule. For example, if the molecule is iron, MRI may be used to quantify the level of the molecule.

In another example if determining the presence of ferritin or more preferably CSF ferritin, the level of the ferritin or more preferably CSF ferritin could be determined through ELISA techniques utilising a secondary detection reagent such as a tagged antibody specific for ferritin. To enhance the accuracy, the CSF sample taken from the patient may be pre-processed prior to detecting iron levels to remove other non-iron binding molecules, or other iron-binding molecules except ferritin. Hence the sample may be treated prior to assessment.

In a non-limiting example where the iron binding molecule is protein, the level of protein can also be detected by an immunoassay. An immunoassay would be regarded by one skilled in the art as an assay that uses an antibody to specifically bind to the antigen (i.e. the protein). The immunoassay is thus characterised by detection of specific binding of the proteins to antibodies. Immunoassays for detecting proteins may be either competitive or non-competitive. Non-competitive immunoassays are assays in which the amount of captured analyte (i.e. the protein) is directly measured. In competitive assays, the amount of analyte (i.e. the protein) present in the sample is measured indirectly by measuring the amount of an added (exogenous) analyte displaced (or competed away) from a capture agent (i.e. the antibody) by the analyte (i.e. the protein) present in the sample.

In one example of a competition assay, a known amount of the (exogenous) protein is added to the sample and the sample is then contacted with the antibody. The amount of added (exogenous) protein bound to the antibody is inversely proportional to the concentration of the protein in the sample before the exogenous protein is added. In another assay, for example, the antibodies can be bound directly to a solid substrate where they are immobilized. These immobilised antibodies then capture the protein of interest present in the test sample. Other immunological methods include but are not limited to fluid or gel precipitation reactions, immunodiffusion (single or double), agglutination assays, immunoelectrophoresis, radioimmunoassays (RIA), enzyme-linked immunosorbent assays (ELISA), Western blots, liposome immunoassays, complement-fixation assays, immunoradiometric assays, fluorescent immunoassays, protein A immunoassays or immunoPCR.

Ferritin can be measured conveniently by means of an enzyme-linked immunosorbent assay (ELISA) or any method available to the skilled addressee.

Hence the brain iron levels that are capable of providing an indication of an individual having or likely to develop cognitive deterioration leading to disorders such as AD, can be measured by any methods available to the skilled addressee preferably by measuring ferritin, most preferably CSF ferritin.

CSF ferritin is measured in CSF samples obtained from cerebral spinal fluid usually by lumbar puncture (spinal tap). As an example, CSF can be collected into polypropylene tubes or syringes and then be transferred into polypropylene transfer tubes without any centrifugation step followed by freezing on dry ice within 1 hour after collection. They may be analysed immediately, or frozen at −80° C. CSF ferritin protein levels were determined using Myriad Rules Based Medicine platform (Human Discovery MAP, v1·0)

The brain iron levels may be measured using any available measurement technology capable of specifically determining the levels of the brain iron from a subject or individual to be tested. The measurement may be either quantitative or qualitative, so long as the measurement is capable of indicating whether the level of brain iron is above or below a reference value from a reference sample.

In another preferred embodiment, the level of brain iron is determined by MRI, optionally ultra field 7T MRI or clinical 3T MRI imaging.

Three main methods exist to quantify iron in vivo with MRI. 1) T2* map: The presence of iron disturbs locally the coherent spins of protons, shortening T2*, which can be imaged with T2* mapping (using multiple gradient echoes, GRE). 2) QSM: Iron presence affects the susceptibility of tissues that can be mapped also using gradient echo imaging. 3) Field-Dependent Relaxation Rate Increase (FDRI): By using T2w collected at two different field strengths (3T & 7T), iron levels may be estimated.

While a considerable literature has developed reporting cross-sectional increases in cortical iron in AD (see below) and other diseases using MRI at ≤3T, there have been caveats concerning the ability of MRI to discriminate iron accumulation from other tissue changes 7T has major advantages over 3T for inferring iron content. One is higher signal to noise ratio, which can be used to increase spatial resolution and/or to reduce scanning time. 7T has the additional benefit of increased sensitivity to magnetic susceptibility. As field strength increases, the contrast in iron-sensitive images is enhanced. This has been demonstrated in gradient echo phase images. Susceptibility-sensitivity combined with the increases in resolution has led to the use of 7T to quantify iron in neurodegenerative diseases such as AD40-42 Parkinson's disease, and amyotrophic lateral sclerosis. Studies have shown enhanced visualisation of the hippocampus and cortical layers, attributed to increased iron sensitivity of 7T. The expected increased sensitivity to iron at 7T may reduce variance and improve statistical power. The higher spatial resolution of 7T over 3T allows for visualisation of cortical layering in the phase, facilitating investigation into iron deposition between cortical layers.

Over the last 20 years, MRI has been used to measure brain iron content, revealing iron elevation in the ageing brain, and that is exaggerated in AD. In cross sectional studies, an inverse correlation exists between brain iron concentration and memory functions in subjectively impaired individuals and individuals with AD, however there has not been a longitudinal study on the impact of iron measured by MRI on AD outcomes. Applicants now show that that high brain iron content translates to an earlier age on onset.

Based on the finding that high brain iron content relative to a reference level, as preferably measured via CSF ferritin, translates to cognitive deterioration, it is considered in the present invention that an increase in brain iron and CSF ferritin would translate to a difference between the patient and the reference level. This difference assists in deducing a risk for cognitive deterioration.

A difference in brain iron level which is an elevation between the patient and the reference level would indicate an increased risk of cognitive deterioration. The degree of elevation will provide an indication of whether there is a diagnosis or an assessment of risk for cognitive deterioration. A small elevation may indicate a risk whereas a high elevation is likely to indicate cognitive deterioration. An increasing elevation between the patient and the reference level will indicate an increased risk for cognitive deterioration.

For the purpose of brevity, some of the description contained herein will be made in the context of AD. It is considered however that the skilled addressee would be capable of understanding that the present invention may also be used as a prognostic or diagnostic or in aiding in the diagnosis/prognosis and/or monitoring of the progression of other neurological disorders such as but not limited to multiple sclerosis, cerebral palsy, Parkinson's disease, neuropathy (conditions affecting the peripheral nerves), dementia, dementia with Lewy bodies (DLB), multi-infarct dementia (MID), vascular dementia (VD), schizophrenia and/or depression, cognitive impairment and frontal temporal dementia.

In another aspect of the invention there is provided a method of diagnosing cognitive deterioration in a patient said method comprising:
determining a first level of brain iron in a patient;
comparing the first level of brain iron to a reference level of brain iron;
determining a difference between the first level of brain iron and the reference level;
deducing cognitive deterioration in the patient from the difference.

The finding by the applicants that high brain iron load is associated with poorer cognition can be used to diagnose cognitive deterioration. A difference in brain iron level which is an elevation between the patient level and the reference level would indicate a diagnosis of cognitive deterioration. The degree of elevation will provide an indication of the severity of cognitive deterioration. A small elevation may indicate a risk whereas a high elevation is likely to indicate a diagnosis of cognitive deterioration. An increasing elevation between the patient and the reference level will indicate an increased cognitive deterioration.

A diagnosis would be understood by one skilled in the art to refer to the process of attempting to determine or identify a possible disease or disorder, and to the opinion reached by this process.

Moreover, a positive diagnosis of cognitive deterioration in a patient can be validated or confirmed if warranted, such as determining the amyloid load or amyloid level to confirm the presence of high neocortical amyloid. The terms amyloid load or amyloid level, often used interchangeably, or presence of amyloid and amyloid fragments, refers to the concentration or level of cerebral amyloid beta (Aβ or amyloid-β) deposited in the brain, amyloid-beta peptide being the major constituent of (senile) plaques.

A patient can also be confirmed as being positive for cognitive deterioration using imaging techniques including, PET and MRI, or with the assistance of diagnostic tools such as PiB when used with PET (otherwise referred to as PiB-PET). Preferably, the patient positive for cognitive deterioration is PiB positive. More preferably, the patient has a standard uptake value ratio (SUVR) which corresponds with high neocortical amyloid load (PiB positive). For instance, current practice regards a SUVR can reflect 1.5 as a high level in the brain and below 1.5 may reflect low levels of neocortical amyloid load in the brain. A skilled person would be able to determine what is considered a high or low level of neocortical amyloid load. As would be appreciated by one of skill in the art, a patient can also be confirmed as being positive for a neurological disease by measuring amyloid beta and tau from the CSF.

Furthermore, in characterising the diagnostic capability of brain iron, preferably ferritin or more preferably CSF ferritin one of skill in the art may calculate the diagnostic cut-off for these biomarkers. This cut-off may be a value, level or range. The diagnostic cut-off should provide a value level or range that assists in the process of attempting to determine or identify a cognitive deterioration.

For example, the level of brain iron, preferably ferritin or more preferably CSF ferritin may be diagnostic for cognitive deterioration if the level is above the diagnostic cut-off. Alternatively, as would be appreciated by one of skill in the art, the level of brain iron, preferably ferritin or more preferably CSF ferritin may be diagnostic for cognitive deterioration if the level is below the diagnostic cut-off.

The diagnostic cut-off for brain iron, preferably ferritin or more preferably CSF ferritin can be derived using a number of statistical analysis software programs known to those skilled in the art. As an example common techniques of determining the diagnostic cut-off include determining the mean of normal individuals and using, for example, +/−2 SD and/or ROC analysis with a stipulated sensitivity and specificity value. Typically a sensitivity and specificity greater than 80% is acceptable but this depends on each disease situation. The definition of the diagnostic cut-off may need to be rederived if used in a clinical setting different to that in which the test was developed. To achieve this control individuals are measured to determine the mean +/−SD. As one of skill in the art would appreciate, using +/−2 SD outside or away from the measurement obtained from control individuals can be used to identify individuals outside of the normal range. Individuals outside of the normal range can be considered positive for disease. The values obtained in a new clinical setting would then be compared to the historic values to determine if the old diagnostic criteria are still applicable as judged by a statistical test. Individuals known to have the disease condition would also be included in the analysis. In situations where both the disease and control state samples are available ROC analysis method with a chosen sensitivity and specificity may be chosen, typically 80%, to determine the diagnostic value that indicates cognitive deterioration. The determination of the diagnostic cut-off can also be determined using statistical models that are known to those skilled in the art.

It would be contemplated that the use of brain iron, preferably ferritin or more preferably CSF ferritin in the methods of the present invention could also be used in combination with other methods of clinical assessment of a neurological disease known in the art in providing a prognostic evaluation of the presence of a neurological disease.

The definitive diagnosis can be validated or confirmed if warranted, such as through imaging techniques including, PET and MRI, or for instance with the assistance of diagnostic tools such as PiB when used with PET (otherwise referred to as PiB-PET).

In applying the methods of the present invention, it is considered that a clinical or near clinical determination regarding the presence of cognitive deterioration in a patient can be made and which may or may not be conclusive with respect to the definitive diagnosis.

Similarly, the methods of the present invention can be used in providing assistance in the prognosis of cognitive deterioration and would be considered to assist in making an assessment of a pre-clinical determination regarding the presence, or nature, of cognitive deterioration. This would be considered to refer to making a finding that a mammal has a significantly enhanced probability of developing cognitive deterioration.

It would be understood by one skilled in the art that clinical determinations for the presence of cognitive deterioration in combination with the assessment of the levels of brain iron, preferably ferritin or more preferably CSF ferritin (in conjunction with information regarding APOE genotype, CSF tau, Aβ and ApoE levels) would be considered to relate to assessments that include, but are not necessarily limited to, memory and/or psychological tests, assessment of language impairment and/or other focal cognitive deficits (such as apraxia, acalculia and left-right disorientation), assessment of impaired judgment and general problem-solving difficulties, assessment of personality changes ranging from progressive passivity to marked agitation. It would be contemplated that the methods of the present invention could also be used in combination with other methods of clinical assessment of a neurological disease known in the art in providing a prognostic evaluation of the presence of a neurological disease.

The definitive diagnosis of cognitive deterioration of a patient suspected of cognitive deterioration can be validated or confirmed if warranted, such as through imaging techniques including, PET and MRI, or for instance with the assistance of diagnostic tools such as PiB when used with PET (otherwise referred to as PiB-PET). Accordingly, the methods of the present invention can be used in a pre-screening or prognostic manner to assess a patient for cognitive deterioration, and if warranted, a further definitive diagnosis can be conducted with, for example, PiB-PET.

In yet another aspect of the present invention there is provided a method for monitoring progression of cognitive deterioration in a patient, said method comprising:
determining a level of brain iron in the patient at first time point;
determining a level of brain iron at in the same patient at a second time point which is after the first time point;
optionally comparing the levels of brain iron from the first and second time points to a reference level;
determining a difference in the levels of brain iron at each of the first and second time points;
deducing progression of cognitive deterioration from the difference in brain iron levels from the first and the second time points.

The changes in the levels of brain iron can additionally be used in assessing for any changes in cognitive deterioration of a patient. Accordingly, in the monitoring of the levels of brain iron, it is possible to monitor for the presence of cognitive deterioration over a period of time, or to track cognitive deterioration progression in a patient.

Accordingly, changes in the level of brain iron from a patient can be used to assess cognitive function and cognitive deterioration, to diagnose or aid in the prognosis or diagnosis of cognitive deterioration and/or to monitor progression toward AD in a patient (e.g., tracking progression in a patient and/or tracking the effect of medical or surgical therapy in the patient).

It may be contemplated to also relate to an altered level relative to a sample previously taken for the same mammal. Hence, there may not be a requirement to compare against a reference level such as from a CN sample. In this regard, a reference level may be the level of brain iron at an earlier time point.

It is contemplated that levels for brain iron can also be obtained from a patient at more than one time point. Such serial sampling would be considered feasible through the methods of the present invention related to monitoring progression of cognitive deterioration in a patient. Serial sampling can be performed on any desired timeline, such as monthly, quarterly (i.e., every three months), semi-annually, annually, biennially, or less frequently. The comparison between the measured levels and predetermined levels may be carried out each time a new sample is measured, or the data relating to levels may be held for less frequent analysis.

In another embodiment, the difference in brain iron level is an elevation between the first and second time points such that the iron levels in the second time point are higher than the first time point relative to the reference level thereby indicating an increased progression of cognitive deterioration. Applicants have shown that patients with comparatively low ferritin (<6.6 ng/ml) will not deteriorate in the foreseeable future. This may potentially explain why 30% of ε4+ve subjects do not develop AD. Conversely, each unit increase of ferritin above this threshold predicted more rapid deterioration.

The methods of the invention can additionally be used for monitoring the effect of therapy administered to a mammal, also called therapeutic monitoring, and patient management. Changes in the level of brain iron, preferably ferritin or more preferably CSF ferritin can be used to evaluate the response of a patient to drug treatment. In this way, new treatment regimens can also be developed by examining the levels of brain iron, preferably ferritin or more preferably CSF ferritin in a patient following commencement of treatment.

A CSF sample may be pre-processed prior to assessment for ferritin levels to remove unbound iron.

The method of the present invention can thus assist in monitoring a clinical study, for example, for evaluation of a certain therapy for a neurological disease. For example, a chemical compound can be tested for its ability to normalise the level of brain iron, preferably ferritin or more preferably CSF ferritin in a patient having cognitive deterioration to levels found in controls or CN patients. In a treated patient, a chemical compound can be tested for its ability to maintain the levels of brain iron, preferably ferritin or more preferably CSF ferritin at a level at or near the level seen in controls or CN patients.

In another embodiment of the invention the method for determining cognitive deterioration further includes:
determining an apolipoprotein E (ApoE) level in the patient;
comparing the level of Apo E in the patient to a reference level of Apo E;
determining a correlation between the Apo E levels in the patient and the reference level to the brain iron levels corresponding to the patient and the reference level of brain iron; and
deducing a risk of cognitive deterioration from the correlation between the Apo E levels and the brain iron levels.

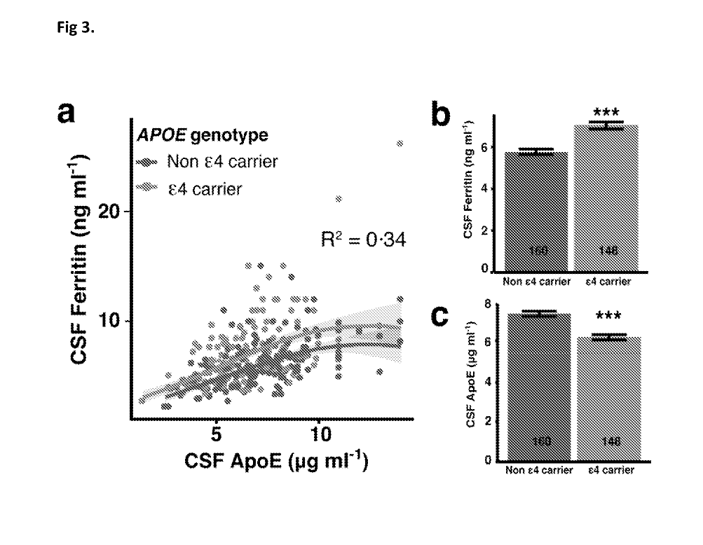

Applicants have found that CSF ferritin levels formed a remarkable association with CSF ApoE levels (FIG. 3a) and subjects with APOE ε4 isoform have elevated CSF ferritin compared to subjects without the AD risk allele (FIG. 3b). Analysis of ApoE and ferritin mRNA levels in post mortem prefrontal cortex confirm an association of similar strength and direction to this CSF protein study (corrected for age, genotype unknown). Measurement of brain iron content in APOE ε3 and ε4 knock-in mice also revealed that mice with ε4 knocked-in had elevated iron compared to WT (+32%; mice aged 3 months;).

Notably, the iron-accumulation mutation of HFE (that causes hemochromatosis) has an epistatic interaction with APOE ε4 to increase AD risk and accelerates disease onset by 5.5 years. Applicants show that APOE ε4 impacts on the association between CSF ferritin and cognitive presentation. In a mixed effects model of longitudinal memory performance (RAVLT; 7 years), elevated CSF ferritin predicted accelerated cognitive decline in APOE ε4 carriers (p=0.003), but not non-carriers (FIG. 5). Thus, harbouring the APOE ε4 allele causes elevation to brain iron, and increased vulnerability toward iron mediated damage as measured using CSF ferritin as a reporter of brain iron status.

Applicants also show that CSF ferritin combines with established AD risk variables, APOE-ε4, CSF tau/Aβ₁₋₄₂ and ApoE, in predicting cognitive decline in normal people over 7 years.

Hence these findings by the applicants can be applied to improve the method for assessing cognitive deterioration. In a preferred embodiment, cognitive deterioration is determined by measuring brain iron using CSF ferritin. From these findings, patients carrying the APOE ε4 allele and high iron are predisposed to cognitive deterioration.

In a further embodiment, the brain iron or CSF ferritin levels may be combined with established AD risk variables such as but not limited to APOE-ε4, CSF tau/Aβ₁₋₄₂ and ApoE, in predicting cognitive decline in normal people.

Accordingly, a positive correlation between brain iron and APOE ε4 allele may indicate an increased risk of cognitive deterioration or decline.

In yet another embodiment, the present method further includes determining a level of a biomarker of cognitive impairment such as but not limited to amyloid β peptides, tau, phospho-tau, synuclein, Rab3a, Aβ and neural thread protein. These additional biomarkers may be used singularly or in combination with the method to assess cognitive deterioration. The methods of the present invention need not be limited to assessing only brain iron, preferably ferritin or more preferably CSF ferritin for determining cognitive deterioration. These additional markers may enhance the accuracy of the method for determining a risk of cognitive deterioration and reduce false positives in the assessment.

In another aspect of the invention there is provided a method for diminishing progression rate of cognitive deterioration, said method comprising lowering brain iron levels.

This method is based on the finding that normal people have worse cognitive performance when they have higher CSF ferritin levels. By measuring the CSF ferritin levels, applicants have correlated the measurements to brain iron and a measure of cognitive deterioration. Without being limited by theory, lowering brain iron, will lower the CSF ferritin levels associated with cognitive deterioration.

In another aspect of the invention there is provided a method for diminishing progression rate of cognitive deterioration, said method comprising lowering CSF ferritin levels.

In yet another aspect of the invention there is provided a method for increasing cognitive performance, said method comprising lowering CSF ferritin levels.

To lower brain iron or CSF ferritin levels compounds such as iron chelators such as Deferiprone may be used. However other compounds that would similarly lower brain iron or CSF ferritin are also included in the scope of the present invention.

The administration of an iron chelator to a patient may reduce the levels of iron in the brain or the CSF in the form of CSF ferritin. This will be particularly effective for patients that show cognitive deterioration. Since high CSF ferritin levels correlate to high brain iron, patients that carry the Apo ε4 allele will also benefit from this treatment. However, CN patients that do not carry the Apo ε4 may also benefit from lowering the brain iron of CSF ferritin levels.

Administration of an iron chelator or an iron lowering drug may be made via any suitable route such as intravenously, subcutaneously, parenterally, orally or topically providing the drug is able to access the area to be treated to lower the iron levels.

Improvements may be determined by methods to assess cognitive deterioration as herein described.

In a further aspect, the present invention provides a kit that can be used for the diagnosis and/or prognosis in a patient for cognitive deterioration or for identifying a patient at risk of cognitive deterioration.

Accordingly, the present invention provides a kit that can be used in accordance with the methods of the present invention for diagnosis or prognosis in a patient for cognitive deterioration or for identifying a patient at risk of cognitive deterioration, or for monitoring the effect of therapy administered to a patient with cognitive deterioration.

The kit as considered can comprise a panel of reagents, that can include, but are not necessarily limited to, polypeptides, proteins, and/or oligonucleotides that are specific for determining levels of brain iron, preferably ferritin or more preferably CSF ferritin. Accordingly, the reagents of the kit that may be used to determine the level brain iron, preferably ferritin or more preferably CSF ferritin to indicate that a subject possesses cognitive deterioration will be capable of use in any of the methods that will detect brain iron, preferably ferritin or more preferably CSF ferritin such as but not limited to 2D DGE, mass spectrometry (MS) such as multiple reaction monitoring mass spectrometry (MRM-MS), Real Time (RT)-PCR, nucleic acid array; ELISA, functional assay, by enzyme assay, by various immunological methods, or by biochemical methods such as capillary electrophoresis, high performance liquid chromatography (HPLC), thin layer chromatography (TLC), hyper-diffusion chromatography, two-dimensional liquid phase electrophoresis (2-D-LPE) or by their migration pattern in gel electrophoreses. For instance, it is envisioned that any antibody that recognises brain iron, preferably ferritin or more preferably CSF ferritin can be used.

In a preferred embodiment, the present invention provides a kit of reagents for use in the methods for the screening, diagnosis or prognosis in a patient for cognitive deterioration, wherein the kit provides a panel of reagents to quantify the level of at least brain iron, preferably ferritin or more preferably CSF ferritin in a sample from a mammal.

In an even further embodiment, the kit further provides means to determine other AD risk variables such as but not limited to APOE-ε4, CSF tau/Aβ1-42 and ApoE for use in combining with the panel of reagents to quantify the level of brain iron, preferably ferritin or more preferably CSF ferritin in a sample from a mammal. The AD risk variables may be determined by quantifying amyloid β peptides, tau, phospho-tau, synuclein, Rab3a, Aβ or neural thread protein. Hence reagents suitable to determine these risk variables may be included in the kit.

A person skilled in the art could use any suitable reagents to determine and quantify the presence of the AD risk variables, APOE-ε4, CSF tau/Aβ₁₋₄₂ and ApoE and more preferably the amyloid β peptides, tau, phospho-tau, synuclein, Rab3a, Aβ and neural thread proteins.

Other aspects of the present invention will become apparent to those ordinarily skilled in the art upon review of the following description of specific embodiments of the invention.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

The present invention will now be more fully described by reference to the following non-limiting Examples.

### EXAMPLES

### Example 1

### Ferritin Levels in the Cerebrospinal Fluid Predict Alzheimer's Disease Outcomes and are Regulated by APOE

Ferritin is the major iron storage protein of the body; by using cerebrospinal fluid (CSF) levels of ferritin as an index, brain iron status impact on longitudinal outcomes was studied in the Alzheimer's Disease Neuroimaging Initiative (ADNI) cohort.

This example shows the association of baseline CSF-ferritin data with biomarker, cognitive, anatomical and diagnostic outcomes over 7 years in the Alzheimer's disease Neuroimaging Initiative (ADNI) prospective clinical cohort. It is shown that CSF ferritin levels have similar utility compared with more established AD CSF biomarkers, the tau/Ab₁₋₄₂ ratio and apolipoprotein E (ApoE) levels, in predicting various outcomes of AD.

(i) Methods

ADNI description. Data were downloaded on 15 Jul. 2014 from the Alzheimer's Disease Neuroimaging Initiative (ADNI) database (adni.loni.usc.edu). The ADNI study has been previously described in detail (Ali-Rahmani et al (2014)).

Recruitment inclusion and exclusion criteria for ADNI 1. Inclusion criteria were as follows: (1) Hachinski Ischaemic Score ≤4; (2) permitted medications stable for 4 weeks before screening; (3) Geriatric Depression Scale score<6; (4) visual and auditory acuity adequate for neuropsychological testing; good general health with no diseases precluding enrolment; (5) six grades of education or work history equivalent; (6) ability to speak English or Spanish fluently; (7) a study partner with 10 h per week of contact either in person or on the telephone who could accompany the participant to the clinic visits.

Criteria for the different diagnostic groups are summarized in Table 1. Groups were age-matched. Cognitively normal (CN) subjects must have no significant cognitive impairment or impaired activities of daily living. Clinical diagnosed AD patients must have had mild AD and had to meet the National Institute of Neurological and Communicative Disorders and Stroke-Alzheimer's Disease and Related Disorders Association criteria for probable AD39, whereas mild cognitive impairment subjects (MCI) could not meet these criteria, have largely intact general cognition as well as functional performance, but meet defined criteria for MCI.

CSF biomarker collection and analysis. CSF was collected once in a subset of ADNI participants at baseline. Ab₁₋₄₂ and tau levels in CSF were measured using the Luminex platform. ApoE and ferritin protein levels were determined using a Myriad Rules Based Medicine platform (Human Discovery MAP, v1.0; see ADNI materials and methods). CSF Factor H (FH) levels were measured using a multiplex human neurodegenerative kit (HNDG1-36K; Millipore, Billerica, Mass.) according to the manufacturer's overnight protocol with minor modifications.

CSF was collected into polypropylene tubes or syringes provided to each site, and then was transferred into polypropylene transfer tubes without any centrifugation step followed by freezing on dry ice within 1 h after collection for subsequent shipment overnight to the ADNI Biomarker Core laboratory at the University of Pennsylvania Medical Center on dry ice. Aliquots (0.5 ml) were prepared from these samples after thawing (1 h) at room temperature and gentle mixing. The aliquots were stored in bar code-labelled polypropylene vials at −80° C. Fresh, never before thawed, 0.5 ml aliquots for each subject's set of longitudinal time points were analysed on the same 96-well plate in the same analytical run for this study to minimize run to run and reagent kit lot sources of variation. Within run coefficient of variation (% CV) for duplicate samples ranged from 2.5 to 5.9% for Ab₁₋₄₂, 2.2-6.3% for tau and the inter-run % CV for CSF pool samples ranged from 5.1 to 14% for Ab1-42, 2.7-11.2% for tau.

Apolipoprotein E (ApoE) and ferritin protein levels were determined using Rules Based Medicine (Human Discovery MAP, v1.0). Further information on the procedures and standard operating procedures can be found in previous publications (Shaw, L. M., et al (2011) and McKhann, G., et al. (1984)) and online (http://www.adni-info.org/).

Structural MRI acquisition and processing. Subjects with a 1.5-T MRI and a sagittal volumetric 3D MPRAGE with variable resolution around the target of 1.2 mm isotopically were included in the analysis. See (www.loni.ucla.edu/ADNI) and for detail (Shaw, L. M., et al (2009)). The hippocampal and ventral volumes utilized were those in the ADNIMERGE primary table as part of the ADNIMERGE R package, downloaded on the 15 Jul. 2014. Only CN and MCI subjects were included in the MRI analysis. MRI scans were performed at baseline, 6 months, 1 year and then yearly for six years.

Statistical analysis. All statistical work was conducted with R (version 3.1.0) (Jack, C. R., Jr., et al. (2008)) using packages ggplot2 (Team, R. C. R: (2014)), nlme (Wickham, H. (2009)), car (Pinheiro, J., Bates, D., DebRoy, S., Sarkar, D. & Team, R. C. (2014)) and Deducer (Fox, J. & Weisberg, S. (2011)). The conditions necessary to apply the regression models, normal distribution of the residuals and the absence of multicollinearity were tested. All models satisfied these conditions. Minimal models were obtained via step down regression using Akaike information criterion (AIC), and Bayesian information criterion (BIC), ensuring that the central hypotheses were maintained. Subjects were excluded from analysis if they had one or more covariates missing. Where subjects prematurely left the study, their data were included in modelling to the point at which they left. The following variables were natural log-transformed to ensure normality: CSF ferritin, Factor H, tau and haemoglobin, while ADAS-cog13 was square-root transformed.

ANCOVA models assessing the differences in each of the CSF biomarkers across the diagnostic groups initially contained age, gender, BMI, APOE genotype, and levels of CSF haemoglobin (Hb) and Factor H. CSF Hb was included as a proxy for blood contamination, to control for the possibility of a traumatic tap introducing plasma ferritin into the CSF samples. FH was used to control for inflammation, since ferritin levels are known to be elevated in certain inflammatory conditions.

Multiple regression models of CSF ferritin and ApoE initially contained age, gender, BMI, APOE genotype, and levels of CSF haemoglobin (Hb) and Factor H, plus various inclusions of CSF tau, Ab₁₋₄₂ and either ferritin or ApoE. The minimal models are described in the table legend of Table 5.

Associations between the baseline Alzheimer's Disease Assessment Scale Cognition (ADAS-cog13) and Rey Verbal Learning Test (RVLT) scores with CSF ferritin, the CSF tau/Ab₁₋₄₂ ratio and CSF ApoE were tested with a covariate adjusted multiple regression for each cognitive scale. For these analyses, age, gender, BMI, years of education, APOE-ε4 allele and baseline diagnosis were initially included as covariates. To assess the association of baseline CSF ferritin levels with the longitudinal clinical outcomes (ADAS-cog13 and RVLT scores over 7 years), linear mixed effects models were used. These models were adjusted for the same variables as the baseline models of cognition, and additionally included time as interacting variable with each of the CSF biomarkers. A significant value for any of these interaction terms would indicate that the variable affected the rate of cognitive change. For the ADAS-cog13, longitudinal analysis, the minimal model included years of education, gender and APOE-ε4 allele. For the longitudinal analysis with RVLT, the minimal model included years of education and gender.

Cox proportional hazards model was used to assess the impact of CSF analytes on the time to AD conversion. The initial model contained age at baseline, gender, years of education and APOE-ε4 genotype as confounding variables together with CSF ApoE, tau/Ab₁₋₄₂ and ferritin. A minimal model containing only the CSF biomarkers was identified via BIC step down procedure and log likelihood test. Logistic regression analysis was used to assess the impact of CSF analytes on risk of conversion to AD. Combinations of CSF ferritin, ApoE and tau/Ab₁₋₄₂ analytes were included in logistic regression models of MCI conversion to AD that were adjusted for age at baseline, gender, years of education, APOE genotype and BMI. These models determined the predictive performance of these analytes in identifying stable MCI participants from MCI participants who, up to 102 months, had a diagnosis change to AD. The receiver-operator curves and the area under the curve were derived from the predictive probabilities of the logistic regression models.

The relationships between CSF ferritin, ApoE, tau/Ab₁₋₄₂ with longitudinal structural (MRI) changes to hippocampus and lateral ventricle were analysed using linear mixed models adjusted for age, years of education, BMI, gender and APOE genotype and intracranial volume. For all models, CSF ferritin, ApoE, tau/Ab₁₋₄₂ and baseline diagnosis were included as fixed effects and were not removed from a minimal model. Two random effects were also included, intercepts and slope (time). An interaction between time and diagnosis, time and CSF ferritin, time and CSF ApoE, as well as time and CSF tau/Ab₁₋₄₂ were also included for all models.

All the AD subjects were excluded from MRI analyses due to low numbers and short follow-up. PET imaging data from ADNI were not included in the analysis because there were too few patients who had CSF ferritin measured and who also underwent PET imaging at baseline.

(ii) Results

The relationship between CSF ferritin and biomarkers of AD. In agreement with other reports, CSF ferritin levels were not different between cognitively normal (CN; n=91), mild cognitive impairment (MCI; n=144) and AD (n=67) subjects (ANCOVA: P=0.591; Table 4) in the ADNI cohort.

TABLE 4
Baseline characteristics of subjects from the ADNI cohort used in this
study, stratified by diagnosis.

| | Units | CN | | MCI | | AD | | p |
|---|---|---|---|---|---|---|---|---|
| n | — | 91 | | 144 | | 67 | | NA |
| Age | Years (S.D.) | 75 · 74 | (5 · 43) | 74 · 85 | (7 · 2) | 74 · 57 | (7 · 61) | 0 · 502 |
| Female | n (%) | 46 | (50 · 55) | 47 | (32 · 64) | 29 | (43 · 28) | 0 · 021 |
| Education | Years (S.D.) | 15 · 67 | (2 · 94) | 15 · 91 | (2 · 95) | 15 · 01 | (2 · 96) | 0 · 123 |
| APOE-ε4 +ve | n (%) | 22 | (24 · 18) | 75 | (52 · 08) | 46 | (68 · 66) | 6 · 50 × 10⁻⁸ |
| ADAS-Cog13 | Units (S.D.) | 9 · 51 | (4 · 16) | 19 · 19 | (5 · 94) | 29 · 22 | (8 · 21) | 2 · 75 × 10⁻⁵⁶ |
| CSF Ferritin | ng/ml (S.D.) | 6 · 4 | (2 · 07) | 6 · 95 | (2 · 72) | 6 · 94 | (2 · 99) | 0 · 591 |
| CSF ApoE | μg/ml (S.D.) | 7 · 3 | (2 · 21) | 7 · 1 | (2 · 22) | 6 · 35 | (2 · 27) | 0 · 012 |
| CSF tau | pg/ml (S.D.) | 69 · 78 | (28 · 01) | 104 · 3 | (52 · 41) | 122 · 63 | (57 · 47) | 4 · 57 × 10⁻⁷ |
| CSF ptau | pg/ml (S.D.) | 24 · 77 | (13 · 34) | 36 · 39 | (16 · 09) | 41 · 39 | (20 · 76) | 1 · 13 × 10⁻⁶ |
| CSF Aβ₁₋₄₂ | pg/ml (S.D.) | 205 · 31 | (56 · 38) | 161 · 06 | (52 · 06) | 142 · 16 | (36 · 84) | 2 · 29 × 10⁻⁶ |
| CSF tau/Aβ₁₋₄₂ | Units (S.D.) | 0 · 39 | (0 · 26) | 0 · 75 | (0 · 5) | 0 · 94 | (0 · 52) | 7 · 80 × 10⁻⁹ |
| Hippocampus | mm³ (S.D.) | 7219 · 6 | (848 · 6) | 6230 · 9 | (1047 · 8) | 5766 · 6 | (1283 · 2) | 6 · 71 × 10⁻²⁰ |
| Lateral | mm³ (S.D.) | 34052 · 62 | (16528 · 1) | 44842 · 52 | (23574 · 05) | 49902 · 53 | (26896 · 68) | 3 · 35 × 10⁻⁵ |
| Ventricle | | | | | | | | |

CN—cognitively normal;
MCI—mild cognitive impairment;
AD-Alzheimer's disease. Unadjusted unit values are presented in the table. p values presented for ANCOVA models of CSF analytes and MRI brain structure was adjusted for age, gender, years of education, BMI, APOE genotype, CSF hemoglobin and CSF Factor H. Intracranial volume was also included in ANCOA models of brain structure.

Neither were there changes in ferritin levels when the cohort were separated according to CSF Ab₁₋₄₂ levels (192 ng l⁻¹ cut-off; as proposed previously in Mattsson, N., et al. (2014)) to reflect likely cerebral amyloid burden (ANCOVA: P=0.946). But in multiple regression modelling of ferritin including the established CSF biomarkers of AD17 (tau, p-tau, Ab₁₋₄₂), CSF ferritin levels were predicted by Ab₁₋₄₂ (partial R²=0.013, P=0.029) and tau (partial R²=0.086, P<0.001; model 1, Table 1), although not by p-tau.

TABLE 1
Modeling of the relationships between CSF ferritin and CSF biomarkers of Alzheimer's disease.

| | Aβ₁₋₄₂ | | | tau | | | ApoE | | | ApoE² | | | | |
|---|---|---|---|---|---|---|---|---|---|---|---|---|---|---|
| Model | β | pR² | p-value | β | pR² | p-value | β | pR² | p-value | β | pR² | p-value | AIC | BIC |
| M1 | 0.051 | 0 · 013 | 0 · 029 | 0.129 | 0 · 086 | 4 · 12 × 10⁻⁸ | — | — | — | — | — | — | 160 | 189 · 5 |
| M2 | 0.003 | 0 · 000 | 0 · 904 | 0.026 | 0 · 003 | 0 · 219 | 0.213 | 0 · 236 | 7.69 × 10⁻²² | 0.045 | 0 · 028 | 0.0004 | 95 · 62 | 121 · 4 |
| M3 | — | — | — | — | — | — | 0.224 | 0 · 341 | 4.04 × 10⁻²⁹ | 0.047 | 0 · 049 | 0.0002 | 93 · 32 | 111 · 7 |

Presented are three models to explore the associations between CSF ferritin levels and the two established CSF biomarkers, Aβ1-42 and tau (M1 and M2), as well as the association between CSF ferritin levels and the newer candidate CSF biomarker, ApoE protein level (M2 & M3).
All models initially contained the variables: age, gender, BMI, APOE genotype, baseline diagnosis, and levels of CSF tau, p-tau, Aβ₁₋₄₂, Hb and FH.
M2 & M3 additionally included ApoE CSF levels.
M1 minimal model contained: APOE genotype, tau, BMI, gender, and FH.
M2 minimal model contained: APOE genotype and ApoE levels, and tau and Aβ₁₋₄₂ were retained
M3 minimal model contained the same as M2, but tau and Aβ₁₋₄₂ were dropped.
AIC—Akaike information criterion,
BIC—Bayesian information criterion.

Since the apolipoprotein E gene (APOE) alleles are the major genetic risk for AD (Corder, E. H., et al. (1993)) and CSF apolipoprotein E protein (ApoE) levels are associated with Ab₁₋₄₂ (Cruchaga, C., et al. (2012); Martinez-Morillo, E., et al. (2014)) and tau (Toledo, J. B., et al. (2014): Martinez-Morillo, E., et al. (2014)) the model was re-built to include CSF ApoE levels. This abolished the relationship between ferritin and the other biomarkers (Ab₁₋₄₂: R²<0.001, P=0.904; tau: R²=0.003, P=0.219; model 2, Table 1). This led to detecting a surprisingly strong relationship between ApoE and ferritin (linear term partial R²=0.243, P=7.69×10⁻²²), which was improved when Ab₁₋₄₂ and tau (non-significant terms) were removed from the model (linear term partial R²=0.341, P=1.52; model 3, Table 1, FIG. 3a).

In model 3, APOE genotype strongly influenced CSF ferritin (P=1.10×10⁻⁸), with the major AD risk allele, ε4, inducing 22% higher levels than non-ε4 carriers (FIG. 3b). Reciprocally, in multiple regression modelling of CSF ApoE, APOE ε4-positive subjects had lower ApoE levels (−16%; P=2.50×10⁻⁰⁹) compared with non-ε4 carriers (FIG. 3c). Plasma ferritin levels were not associated with plasma ApoE levels or APOE ε4 allele status, but there was a modest association between plasma ferritin and CSF ferritin levels (β=0.075, P=0.0002).

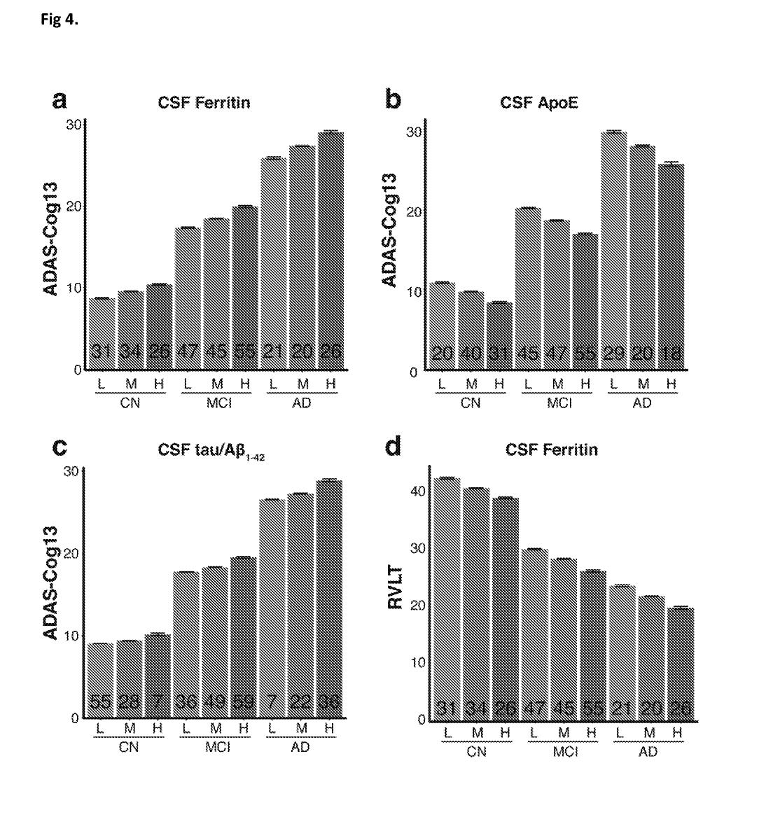

Association of ferritin with neuropsychiatric assessments. The relationship of CSF ferritin and cognitive performance in AD was examined. Baseline ADAS-Cog13 (The Alzheimer's Disease Assessment Scale) score was associated with CSF ferritin (P=0.006; Table 5), ApoE levels (P=0.0003) and tau/Ab₁₋₄₂ ratio (P=0.025), independently, in a multiple regression model containing the AD biomarkers and other clinical variables. In tertile analysis, high (47.2 ng m⁻¹), compared with low (<5.4 ng ml⁻¹), levels of ferritin were associated with a ˜3 point poorer ADAS-cog13 score (FIG. 4a). Similarly, in tertiles, lower levels of ApoE (FIG. 4b) were associated with a ˜4 point worse ADAS-Cog13, and higher tau/Ab₁₋₄₂ ratio was associated with a ˜2 point worse ADAS-Cog13 (FIG. 4c), as previously reported (Toledo, J. B., et al. (2014): Kester, M. I., et al. (2009)).

To determine whether baseline values of CSF ferritin predict longitudinal cognitive outcome, a mixed effects model of annual ADAS-Cog13 scores over 7 years WAS constructed (Table 5 for statistics, Table 2 for patient numbers) and observed that both ApoE (P=0.006) and tau/Ab₁₋₄₂ ratio (P=2.7×10⁻⁷) were still associated with rate of cognitive change (interacted with time), as previously reported (Toledo, J. B., et al. (2014): Kester, M. I., et al. (2009)). Ferritin, however, impacted on ADAS-Cog13 by a constant cross-sectional decrement (P=4.93×10⁻⁴ main effect only; Table 5).

TABLE 2
Patient numbers for longitudinal cognitive assessment.

| | CN | MCI | AD |
|---|---|---|---|
| Bl | 88 | 137 | 63 |
| 6 m | 88 | 137 | 61 |
| 1 yr | 86 | 138 | 63 |
| 2 yr | 82 | 123 | 52 |
| 3 yr | 78 | 97 | 4 |
| 4 yr | 55 | 47 | 2 |
| 5 yr | 49 | 39 | 0 |
| 6 yr | 54 | 37 | 0 |
| 7 yr | 43 | 27 | 0 |

Bl: Baseline.
CN: cognitively normal.
MCI: Mild cognitive impairment.
AD: Alzheimer's disease

TABLE 5
Modelling the association of CSF biomarkers on AD outcomes.

| Model | Ferritin [symbol] | | tan/Aβ₁₋₄₂ | | ApoE | |
|---|---|---|---|---|---|---|
| Cross-sectional cognition | β | | β | | β | |
| (MR) | (se) | p | (se) | p | (se) | p |
| ADAS-Cog13 [symbol] | 0 · 139 (0 · 050) | 0 · 006 | 0 · 104 (0 · 046) | 0 · 025 | −0 · 178 (0 · 049) | 0 · 0003 |
| RVLT | −1.77 (0.559) | 0 · 0017 | NS | NS | 1 · 033 (0 · 564) | 0 · 0677 |
| Longitudinal cognition | β | | β | | β | |
| (MELM) | (se) | p | (se) | p | (se) | p |
| ADAS-Cog13 [symbol] | | | | | | |
| main effect | 0 · 178 (0 · 051) | 0 · 0005 | 0 · 129 (0 · 049) | 0 · 009 | −0 · 180 (0 · 051) | 0 · 0004 |
| interaction-time | 0 · 0005 (0 · 016) | 0 · 977 | 0 · 081 (0 · 016) | 2 · 70 × 10⁻⁷ | −0 · 044 (0 · 016) | 0 · 006 |
| RVLT | | | | | | |
| main effect | −1 · 60 (0 · 63) | 0 · 012 | −0 · 847 (0 · 608) | 0 · 165 | 1 · 03 (0 · 63) | 0 · 014 |
| interaction-time | −0 · 035 (0 · 152) | 0 · 817 | −0 · 610 (0 · 150) | 4 · 85 × 10⁻⁵ | 0 · 279 (0 · 152) | 0 · 066 |
| MCI conversion to AD | Statistic* | p | Statistic* | p | Statistic* | p |
| Cox (Hazard ratio) | 1 · 10 (1 · 01-1 · 19) | 0 · 030 | 1 · 53 (1 · 03-2 · 28) | 0 · 037 | 0 · 83 (0 · 73-0 · 95) | 0 · 008 |
| LR (Odds ratio) | 2 · 32 (1 · 86-2 · 90) | 8 · 001 × 10⁻²⁵ | 1 · 45 (1 · 16-1 · 80) | 0 · 0001 | 0 · 38 (0 · 30-0 · 48) | 1 · 88 × 10⁻²⁷ |
| Rate of MRI atrophy (MELM) | β (se) | p | β (se) | p | β (se) | p |
| Hippocampus | −18 · 33 (7 · 86) | 0 · 019 | −35 · 31 (7 · 79) | 6 · 81 × 10⁻⁶ | 21 · 38 (8 · 02) | 0 · 008 |
| Lateral ventricles [symbol] | 0 · 007 (0 · 003) | 0 · 008 | 0 · 013 (0 · 002) | 4 · 19 × 10 [symbol] | −0 · 009 (0 · 003) | 0.0002 |

All models initially contained the variables: age, gender, BMI, APOE genotype, baseline diagnosis;
the MRI models additionally included intracranial volume.
Minimal models for the cognition models included baseline diagnosis, gender, years of education and the AD CSF biomarkers.
Minimal model for the Cox proportional hazard model (Cox) [symbol]
indicates data missing or illegible when filed

contained only the AD CSF biomarkers. Minimal models for the MRI models contained age, gender, baseline diagnosis, years of education, APOE ε4 status, and intracranial volume. All subjects with available data were included in the cognition models. Only subjects who were classed as MCI at baseline were included in the MCI conversion models. The MRI models contained subjects who were classed as cognitively normal or MCI at baseline. AD subjects at baseline were not included because of low numbers and lack of follow up (Table 3). *The statistics for the conversion models were based on 1 interquartile range change for each analyte (ferritin: 3.3 ng/ml, tau/Aβ₁₋₄₂: 0.67 units; ApoE: 3.1 μg/ml). ^{†}Ferritin values were log transformed, excluding non-parametric Cox and LR models. ̂The β-coefficient is for the square root of ADAS-Cog13. # For Lateral ventricles, the β-coefficient is for natural log of the ventricle volume. MR: Multiple regression, MELM: Mixed Effects Linear Model. Cox: Cox proportional hazard model. LR: Logistic regression. NS: Not Significant.

Cognition was modelled using the Rey verbal learning test (RVLT), which is more sensitive in distinguishing control and MCI patients. In this model, only ferritin levels were associated with cross-sectional cognitive performance (P=0.0017; Table 5, FIG. 4d), but CSF ferritin was not associated with rate of deterioration in a longitudinal model (P=0.817; Table 5). Baseline tau/Ab₁₋₄₂ ratio (P=4.85×10⁻⁵) was associated with rate of cognitive decline on RVLT, but there was only a trend for ApoE (P=0.066). Hence, in both cognitive scales, CSF ferritin impacted on performance by a constant amount, regardless of disease status.

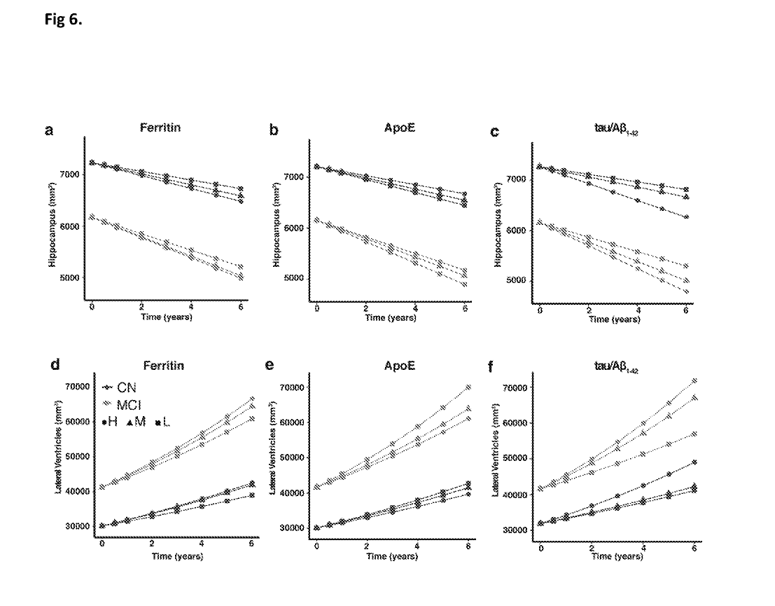

If high ferritin levels worsened the cognitive performance by a constant value over time, then MCI individuals with high ferritin levels would satisfy the criteria for an AD diagnosis at a comparatively earlier interval. To investigate this, a Cox proportional hazards model was employed on 144 MCI subjects who had CSF ferritin, ApoE and tau/Ab₁₋₄₂ measurements. In a minimal model (containing only these CSF biomarkers; Table 5) of MCI conversion over 7 years, ferritin (P=0.03; FIG. 5a), ApoE (P=0.008; Supplementary FIG. 6a) and tau/Ab₁₋₄₂ (P=0.037; Supplementary FIG. 6b) were each significant predictive variables.

Using this model it was estimated how many months was required for 50% survivorship for each quintile of each biomarker. A linear model of these values was constructed (in months; y-axis) against the values for the quintile boundaries of each analyte (in designated units; x-axis). The gradient of these functions estimates the change in mean age of conversion (in months) associated with one unit change in the baseline CSF analyte. For comparison between biomarkers, the change was expressed in mean age of conversion associated with an s.d. change to the analyte value. One s.d. change to ferritin was associated with a 9.5-month shift in age of conversion, compared with 18.2 and 8.6 months for ApoE and tau/Ab₁₋₄₂, respectively (FIG. 5b).

In separate adjusted logistic regression models, an increase in the baseline concentration of each biomarker by its interquartile range increased the odds of converting to AD for ferritin (OR: 1.36, 95% CI: 1.17-1.58) and tau/Ab₁₋₄₂ ratio (OR: 1.13, CI: 0.95-1.35), and decreased the odds for ApoE (OR: 0.72, CI: 0.61-0.85). Including all three analytes into the one model increased the predictive value of each analyte (OR (CI): ferritin=2.32 (1.86-2.9], tau/Ab₁₋₄₂=1.45[1.16-1.8], ApoE=0.38[0.3-0.48]; Table 5).

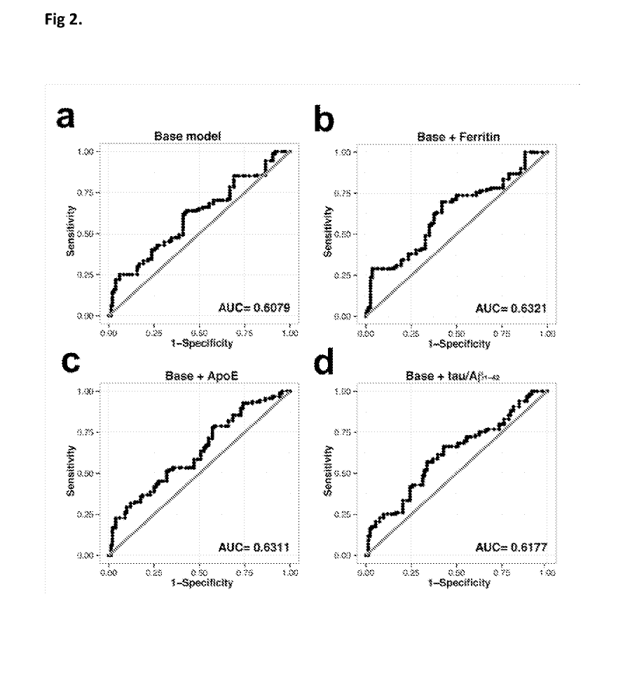

Receiver-operating curves based on the logistic regression models determined the accuracy of these analytes to predict conversion to AD. The area under the curve (AUC) of the base model (age, gender, years of education, BMI, APOE ε4 genotype) was 0.6079 (FIG. 5c), which was increased by the singular inclusions of either ferritin (AUC: 0.6321; FIG. 2b), ApoE (0.6311; FIG. 2c) or marginally by tau/Ab₁₋₄₂ (0.6177; FIG. 2d). When the tau/Ab₁₋₄₂ was included in the model containing ApoE, the AUC increased slightly (from 0.6311 to 0.6483; FIG. 5d). This performance, which combined the established CSF biomarkers for AD, was improved markedly by adding ferritin values (from 0.6483 to 0.6937 FIG. 5e).

Association of ferritin with brain atrophy. It was investigated whether ferritin levels associate with neuroanatomical changes to the hippocampus and lateral ventricular area in yearly intervals over a 6-year period for CN and MCI subjects (Table 3 for patient numbers).

TABLE 3
Patient numbers for longitudinal MRI assessment.

| | CN | MCI | AD |
|---|---|---|---|
| Bl | 79 | 108 | 48 |
| 6 m | 80 | 108 | 49 |
| 1 yr | 74 | 96 | 37 |
| 2 yr | 66 | 85 | 35 |
| 3 yr | 57 | 62 | 0 |
| 4 yr | 38 | 35 | 0 |
| 5 yr | 26 | 24 | 0 |
| 6 yr | 24 | 14 | 0 |

Bl: Baseline.
CN: cognitively normal.
MCI: Mild cognitive impairment.
AD: Alzheimer's disease

The impact of CSF ferritin when the other biomarkers were also included in modelling was explored, whereas CSF ferritin has previously been shown to predict atrophy of various brain structures when considered in isolation. Baseline ApoE, ferritin and tau/Ab₁₋₄₂ values each independently predicted hippocampal volume in an adjusted longitudinal model (Table 5). The rate of atrophy of the hippocampus was greater in individuals with high CSF ferritin (P=0.02; FIG. 6a). Low CSF ApoE (P=0.008; FIG. 6b) or high tau/Ab₁₋₄₂ (P=6.80×10⁻⁶; FIG. 6c) also predicted atrophy. Lateral ventricular enlargement over time was similarly associated independently with high-CSF ferritin (P=0.008; FIG. 6d), low-CSF ApoE (P=0.0002; FIG. 6e), or high Q5 tau/Ab₁₋₄₂ (P=4.19×10⁻⁸; FIG. 6f).

(iii) Discussion

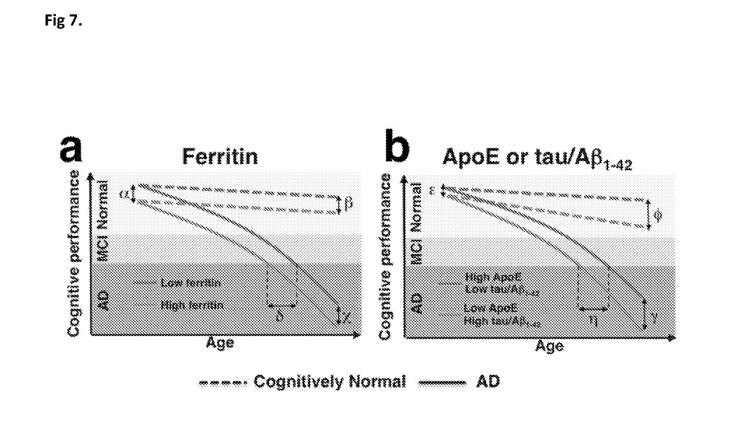

These analyses show that CSF ferritin levels were independently related to cognitive performance in the ADNI cohort and predicted MCI conversion to AD. The magnitude impact of ferritin on these outcomes was comparable to the established biomarkers, ApoE and tau/Ab₁₋₄₂; however, the nature of the effect of ferritin was not the same. Ferritin was associated with constant shift in cognitive performance over the study period (FIG. 7a), whereas the decrements associated with the other biomarkers were exaggerated over time (FIG. 7b). A downward shift (poorer cognitive presentation) in response to high ferritin levels (1.77 RVLT points per 1 ng ml⁻¹ ferritin; Table 5) results in an earlier age of diagnosis (3 months per 1 ng ml⁻¹ ferritin; FIG. 5b). This would be consistent with findings that patients with an early age of AD onset have greater neocortical iron burden than late-onset patients. Collectively these data support consideration of therapeutic strategies that lower brain iron, which have reported beneficial outcomes in Phase II trials of Alzheimer's and Parkinson's diseases. Lowering CSF ferritin as might be expected from a drug like deferiprone, could conceivably delay MCI conversion to AD by as much as 3 years.

This data provides exploratory insights into iron in AD aetiopathogenesis, identifying an unexpected interaction of ApoE with ferritin. That ferritin levels are increased by the APOE-ε4 allele argues that ApoE influences ferritin levels, rather than the reverse. These findings indicate that APOE genotype should influence constitutive brain iron burden.

These data support the concept that APOE ε4 status confers susceptibility to AD by increasing ferritin levels.

This example shows that baseline CSF ferritin levels were negatively associated with cognitive performance over 7 years in 91 cognitively normal, 144 mild cognitive impairment (MCI) and 67 AD subjects, and predicted MCI conversion to AD. Ferritin was strongly associated with CSF apolipoprotein E levels and was elevated by the Alzheimer's risk allele, APOE-ε4. These findings reveal that elevated brain iron adversely impacts on AD progression, and introduce brain iron elevation as a possible mechanism for APOE-ε4 being the major genetic risk factor for AD.

### Example 2

### Cerebrospinal Ferritin Determines the Risk of Cognitive Decline in Pre-Clinical APOE-E4 Carriers

The ε4 allele of apolipoprotein E (APOE) confers the greatest risk for Alzheimer's disease (AD), and recent data implicates brain-iron load as the risk vector since ε4 carriage elevates cerebrospinal (CSF) ferritin ≈20% (Ayton S et al (2015)). CSF ferritin levels predict longitudinal cognitive performance and the risk for Mild Cognitive Impairment (MCI) subjects transitioning to AD. This example shows that CSF ferritin combines with established AD risk variables, APOE-ε4, CSF tau/Aβ₁₋₄₂ and ApoE, in predicting cognitive decline in normal people over 7 years.

(i) Methods

This example used data obtained from the Alzheimer's Disease Neuroimaging Initiative (ADNI) database (adni.loni.usc.edu; 15 Jul. 2014).

Baseline CSF levels of Aβ₁₋₄₂, tau (Luminex), ApoE, ferritin (Myriad Rules Based Medicine) and longitudinal Ray Auditory-Visual Learning Task (RAVLT; sensitive to early changes) and AD Assessment Scale-cognitive subset (ADAS-Cog13) scores were analysed using linear mixed effects models with R (version 3.2.1). Normality and the absence of multicolinearity were confirmed. Data from subjects who left prematurely were included to the point of leaving.

(ii) Results

The initial modelling of pre-dementia subjects (Table 6) revealed two-way interaction between tau/Aβ₁₋₄₂ ratio and time on cognitive performance (RAVLT: P=0.011; ADAS-Cog13: P=0.0011), confirming that this index predicts the rate of cognitive deterioration. Tau/Aβ₁₋₄₂ did not interact with other AD risk factors: APOE-ε4 status, diagnosis, ferritin, or ApoE levels (either separately, or combined in higher-order terms). In contrast, CSF ferritin predicted cognition in a four-way interaction with time, APOE ε4 and diagnosis (RAVLT: P=0.0169; ADAS-Cog13 P=0.0297).

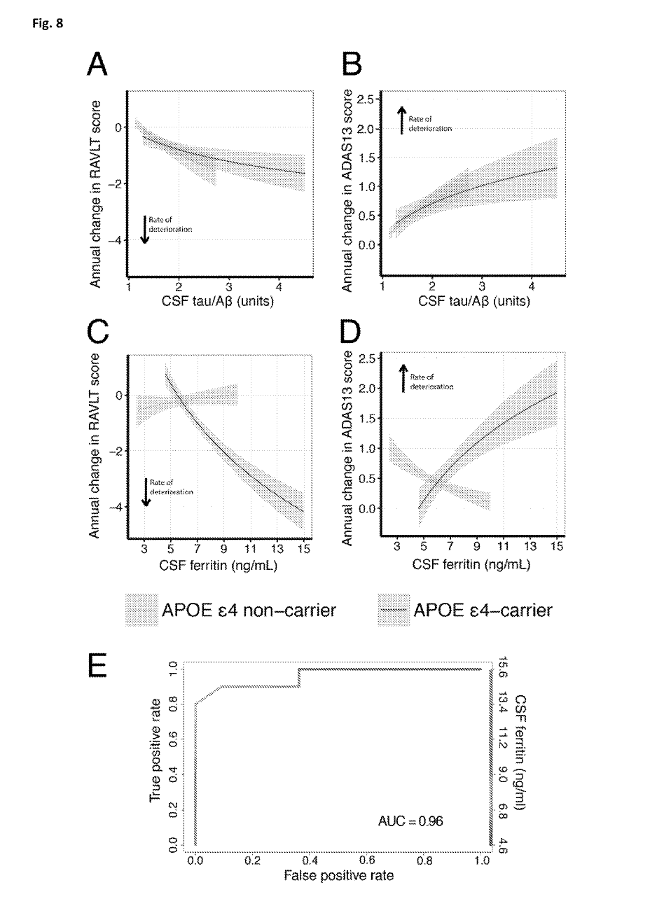

In separate modelling of Cognitively Normal (CN) and MCI subjects, tau/Aβ₁₋₄₂ predicted cognitive deterioration for MCI (RAVLT: P=0.072; ADAS-Cog13; P=0.019) and CN (RAVLT: P=0.039; ADAS-Cog13: P=0.006; FIG. 8A,B) subjects, and this index did not interact with the other included variables.

All interaction terms with ferritin were non-significant for MCI subjects, but there was a significant main effect on cognitive performance (RAVLT: P=0.019; ADAS-Cog13: P=0.042; consistent with prior, simplified modelling as described in Ayton S et al (2015)). For CN subjects, however, ferritin predicted cognitive deterioration in a 3-way interaction with time and ε4 (RAVLT: P=0.0035; ADAS-Cog13: P=0.010; FIG. 8C,D). Categorization of CN subjects according to ε4 status revealed that ferritin strongly predicted cognitive decline in ε4+ve subjects (RAVLT: P=0.0008; ADAS-Cog13: P=0.016). For ε4-ve subjects, lower ferritin levels predicted a modest deterioration in cognition in ADAS-Cog13 (P=0.016) but not in RAVLT (P=0.477).

Finally, baseline CSF ferritin was tested to determine whether it could be used to discriminate stable from declining (≥1 point/year worsening on RAVLT) CN ε4+ve subjects. The area under the Receiver Operating Characteristic (ROC) curve was 0.96, at a threshold predictive value of 6.6 ng ferritin/ml (FIG. 8E).

TABLE 6
Patient demographics and statistical models. Separate covariate-adjusted linear mixed effects linear models of longitudinal (7 year) cognitive
performance (RAVLT, ADAS-Cog13) in CN and MCI subjects (AD subjects were excluded from the longitudinal analysis because of low rate of follow
up). Variables initially included in modelling were: age, gender, BMI, years of education, APOE-ε4 allele, baseline diagnosis, CSF tau/Aβ, CSF
ApoE, CSF ferritin, before minimal models were obtained using Akaike information criterion and Bayesian information criterion.

| | All subjects | | MCI only | | CN only | | CN ε4 negative | | CN ε4 positive | |
|---|---|---|---|---|---|---|---|---|---|---|
| Demographics | n | S.D. or % | n | S.D. or % | n | S.D. or % | n | S.D. or % | n | S.D. or % |
| Subjects | 234 | — | 144 | — | 90 | — | 69 | — | 21 | — |
| APOE ε4 + ve | 96 | 41% | 75 | 52% | 21 | 23% | 0 | 0% | 21 | 100% |
| Age | 75.2 | 6.6 | 74.9 | 7.2 | 75.7 | 5.5 | 75.6 | 5.2 | 76.0 | 6.4 |
| Gender (Female) | 93 | 40% | 47 | 33% | 46 | 51% | 38 | 55% | 8 | 35% |
| Education years | 15.8 | 3.0 | 15.9 | 3 | 15.6 | 3.0 | 15.7 | 2.8 | 15.5 | 3.4 |
| RAVLT | f | P | f | P | f | P | f | P | f | P |
| Controlling variables | | | | | | | | | | |
| Diagnosis | 57.08 | 1.06 × 10\hat{ }−12 | NA | NA | NA | NA | NA | NA | NA | NA |
| Gender | 11.96 | 0.0007 | 2.83 | 0.095 | 16.17 | 0.0001 | 12.91 | 0.0006 | 4.84 | 0.043 |
| Education years | 7.16 | 0.008 | 0.25 | 0.616 | 17.75 | 0.0001 | 15.454 | 0.002 | 3.13 | 0.096 |
| Tesing variable/interaction | | | | | | | | | | |
| tau/Aβ₁₋₄₂ | 1.10 | 0.296 | 1.4.3 | 0.233 | 0.04 | 0.833 | 0.329 | 0.568 | 0.552 | 0.468 |
| tau/Aβ₁₋₄₂ × time | 6.54 | 0.011 | 3.24 | 0.072 | 4.27 | 0.039 | 6.058 | 0.014 | 0.645 | 0.424 |
| ferritin [symbol] | 0.064 | 0.800 | 5.55 | 0.019 | 0.018 | 0.894 | 0.047 | 0.830 | 0.743 | 0.401 |
| ferritin × time × ε4 × | 5.73 | 0.0169 | 0.477 | 0.490 | 8.627 | 0.0035 | 0.507 | 0.477 | 12.05 | 0.0008 |
| diagnosis [symbol] | | | | | | | | | | |
| ADAS-cog13 [symbol] | f | P | f | P | f | P | f | P | f | P |
| Controlling variables | | | | | | | | | | |
| Diagnosis | 112 | <1.0 × 10\hat{ }−26 | NA | NA | NA | NA | NA | NA | NA | NA |
| Gender | 4.07 | 0.0447 | 0.283 | 0.598 | 10.3 | 0.002 | 10.604 | 0.002 | 0.957 | 0.343 |
| Education years | 5.78 | 0.0169 | 1.05 | 0.306 | 9.65 | 0.003 | 13.973 | 0.0004 | 0.002 | 0.862 |
| Testing variable/interaction | | | | | | | | | | |
| tau/Aβ₁₋₄₂ | 2.59 | 0.109 | 2.78 | 0.098 | 0.06 | 0.805 | 0.007 | 0.933 | 0.03 | 0.862 |
| tau/Aβ₁₋₄₂ × time | 10.72 | 0.0011 | 5.00 | 0.026 | 7.61 | 0.006 | 7.630 | 0.006 | 1.829 | 0.180 |
| Ferritin | 1.51 | 0.221 | 4.22 | 0.042 | 1.67 | 0.200 | 1.985 | 0.164 | 1.218 | 0.286 |
| ferritin × time × ε4 × | 4.73 | 0.0297 | 0.237 | 0.627 | 6.69 | 0.010 | 5.858 | 0.016 | 6.044 | 0.016 |
| diagnosis [symbol] | | | | | | | | | | |

NA: Not applicable.
@ ADAS-Cog13 variable was squire-root transformed.
# CSF ferritin was natural log-transformed.
*This interaction variable was simplified to lower order terms when the cohort was restricted according to the column titles.
CN—Cognitively normal; MCI—Mild Cognitive Impairment; RAVLT—Ray Auditory Visual Learning Test; ADAS-Cog13—Alzheimer's disease Rating Scale- cognition. [symbol]
indicates data missing or illegible when filed

(iii) Discussion

These data show that CN ε4+ve subjects with comparatively low ferritin (<6.6 ng/ml) will not deteriorate in the foreseeable future, which could potentially explain why 30% of ε4+ve subjects do not develop AD. Conversely, each unit increase of ferritin above this threshold predicted more rapid deterioration.

These findings reveal a markedly divergent impact of CSF ferritin on ε4 carriers and non-carriers. CSF ferritin levels in ε4 carriers are all ≥4.5 ng/ml, but in non-ε4 subjects range to half that value, whereupon subjects express slight cognitive deterioration (FIG. 8C,D).

### Example 3

### Assessing a Risk of Cognitive Deterioration in a Patient

In conducting the methods of the present invention, it is contemplated that a patient will be assessed for a level of cognitive ability. This level will set a base for determining whether they will over time deteriorate. They patient may already show signs of cognitive impairment after being assessed.

A CSF sample may be obtained and the CSF ferritin level determined by methods such as immunoassay. This sample may then be compared to a predetermined sample from a CN patient processed in the same manner.

A difference in the CSF ferritin levels of the patient and that of the CN patient will be determined. Depending on the degree of difference, the degree of cognitive deterioration can be determined. If the difference is large and the CSF ferritin level of the patient is high relative to the CN patient level, the patient presenting for assessment may show a higher risk of cognitive deterioration. If the difference is small relative to the CN patient level, the patient presenting for assessment may show a lower risk of cognitive deterioration.

This test may be conducted in parallel to determining the genotype of the patient. If the patient carries the Apo ε4 allele, the risk of cognitive deterioration will be higher.

### Example 4

### Monitoring Cognitive Deterioration in a Patient

A patient is tested according to Example 3 at a first time point. A second test is conducted at another time point after the first time point. The difference between the patient CSF ferritin and a reference level from a CN patient is assessed.

This difference may then be compared to the difference from the first time point.

If the difference is greater, the deterioration will have advanced.

The patient may be diagnosed as having cognitive deterioration based in the increasing CSF ferritin levels.

### Example 5

### Diminishing Progression Rate of Cognitive Deterioration in a Patient

A patient is assessed as in Example 3 for the level of cognitive deterioration based on their CSF ferritin levels. Deferiprone is administered to the patient for a time and a dose calculated by the size, age and weight of the patient.

The patient is reassessed for cognitive ability after a time to assess whether cognitive deterioration has been diminished.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

### REFERENCES

Ali-Rahmani, F., Schengrund, C. L. & Connor, J. R. HFE gene variants, iron, and lipids: a novel connection in Alzheimer's disease. Frontiers in pharmacology 5, 165 (2014).
McKhann, G., et al. Clinical diagnosis of Alzheimer's disease: report of the NINCDS-ADRDA Work Group under the auspices of Department of Health and Human Services Task Force on Alzheimer's Disease. Neurology 34, 939-944 (1984).
Shaw, L. M., et al. Qualification of the analytical and clinical performance of CSF biomarker analyses in ADNI. Acta neuropathologica 121, 597-609 (2011).
Shaw, L. M., et al. Cerebrospinal fluid biomarker signature in Alzheimer's disease neuroimaging initiative subjects. Annals of neurology 65, 403-413 (2009).
Jack, C. R., Jr., et al. The Alzheimer's Disease Neuroimaging Initiative (ADNI): MRI methods. Journal of magnetic resonance imaging: JMRI 27, 685-691 (2008).
Team, R. C. R: A Language and Environment for Statistical Computing. (R Foundation for Statistical Computing, 2014).
Wickham, H. ggplot2: elegant graphics for data analysis, (Springer N.Y., 2009).
Pinheiro, J., Bates, D., DebRoy, S., Sarkar, D. & Team, R. C. nlme: Linear and Nonlinear Mixed Effects Models. (2014).
Fox, J. & Weisberg, S. An R Companion to Applied Regression, (Sage, 2011).
Mattsson, N., et al. Effects of cerebrospinal fluid proteins on brain atrophy rates in cognitively healthy older adults. Neurobiology of aging 35, 614-622 (2014).
Corder, E. H., et al. Gene dose of apolipoprotein E type 4 allele and the risk of Alzheimer's disease in late onset families. Science 261, 921-923 (1993).
Cruchaga, C., et al. Cerebrospinal fluid APOE levels: an endophenotype for genetic studies for Alzheimer's disease. Human molecular genetics 21, 4558-4571 (2012).
Martinez-Morillo, E., et al. Total apolipoprotein E levels and specific isoform composition in cerebrospinal fluid and plasma from Alzheimer's disease patients and controls. Acta neuropathologica (2014).
Toledo, J. B., et al. CSF Apo-E levels associate with cognitive decline and MRI changes. Acta neuropathologica 127, 621-632 (2014).
Kester, M. I., et al. CSF biomarkers predict rate of cognitive decline in Alzheimer disease. Neurology 73, 1353-1358 (2009).
Ayton S, Faux N G, Bush A I, Alzheimer's Disease Neuroimaging I. Ferritin levels in the cerebrospinal fluid predict Alzheimer's disease outcomes and are regulated by APOE. Nature communications. 2015; 6:6760.

1. A method for predicting a risk of cognitive deterioration in a patient, said method comprising:
determining a first level of brain iron in a patient;
comparing the first level of iron to a reference level of brain iron;
determining a difference between the first level of brain iron and the reference level; and
deducing a risk for cognitive deterioration in the patient from the difference.

2. A method according to claim 1 wherein the difference in brain iron level is an elevation thereby indicating an increased risk of cognitive deterioration

3. A method of diagnosing cognitive deterioration in a patient said method comprising:
determining a first level of brain iron in a patient;
comparing the first level of brain iron to a reference level of brain iron;
determining a difference between the first level of brain iron and the reference level;
deducing cognitive deterioration in the patient from the difference.

4. A method according to claim 3 wherein the difference in the brain iron level is an elevation thereby diagnosing cognitive deterioration.

5. A method for monitoring progression of cognitive deterioration in a patient, said method comprising:
determining a level of brain iron in the patient at first time point;
determining a level of brain iron at in the same patient at a second time point which is after the first time point;
optionally comparing the levels of brain iron from the first and second time points to a reference level;
determining a difference in the levels of brain iron at each of the first and second time points;
deducing progression of cognitive deterioration from the difference in brain iron levels from the first and the second time points.

6. A method according to claim 5 wherein the difference in brain iron level is an elevation between the first and second time points such that the iron level in the second time point is higher than the first time point relative to the reference level thereby indicating an increased progression of cognitive deterioration.

7. A method according to any one of claims 1 to 6 wherein the levels of brain iron are determined as a measure of an iron related protein level selected from the group including ceruloplasmin, amyloid precursor protein, tau, ferritin, transferrin, transferrin binding protein or by MRI, and sonography.

8. A method according to any one of claims 1 to 7 wherein the brain iron is cortical iron.

9. A method according to any one of claims 1 to 8 wherein the level of brain iron is determined as a measure of cerebrospinal fluid (CSF) ferritin.

10. A method according to any one of claims 1 to 8 wherein the level of brain iron is determined by MRI, optionally ultra field 7T MRI or clinical 3T MRI imaging.

11. A method according to any one of claims 1 to 10 further including:
determining an apolipoprotein E (ApoE) level in the patient;
comparing the level of Apo E in the patient to a reference level of Apo E from a CN individual;
determining a correlation between the Apo E levels in the patient and the reference level to the brain iron levels corresponding to the patient and the reference level in the brain; and
deducing a risk of cognitive deterioration from the correlation between the Apo E levels and the brain iron levels.

12. A method according to claim 11 wherein the correlation is a positive correlation thereby indicating an increased risk of cognitive deterioration.

13. A method according to claim 11 or 12 further including:
determining an Apo E genotype in the patient.

14. A method according to claim 13 wherein the Apo E genotype comprises the Apo ε4 allele.

15. A method according to any one of claims 11 to 14 wherein the Apo E levels are determined as a measure of CSF Apo E levels.

16. A method according to any one of claims 1 to 15 further including determining a level of a biomarker of cognitive impairment selected form amyloid β peptides, Tau, phospho-tau, synuclein, Rab3a, Aβ, CSF tau/Aβ1-42 and neural thread protein, optionally Tau or Aβ.

17. A method according to any one of claims 1 to 16 wherein the reference level is determined from a cognitively normal individual.

18. A method according to any one of claims 1 to 17 wherein the cognitive deterioration includes mild cognitive impairment (MCI), MCI conversion to Alzheimer's Disease (AD), and AD.

19. A method according to any one of claims 1 to 18 wherein prior to measuring brain iron, ferritin or CSF ferritin, unbound cellular iron is removed so that only iron related protein levels are determined.

20. A method for diminishing progression rate of cognitive deterioration in a patient, said method comprising lowering brain iron levels in the patient.

21. A method for diminishing progression rate of cognitive deterioration in a patient, said method comprising lowering CSF ferritin levels in the patient.

22. A method for increasing cognitive performance in a patient, said method comprising lowering CSF ferritin levels in the patient.

23. A method according to claim 21 or 22 wherein the CSF ferritin levels are lowered by administering an effective amount of Deferiprone or an iron lowering drug.

24. A method according to any one of claims 20 to 23 wherein the patient has an Apo E genotype and optionally carries the ε4 allele.

25. A method according to any one of claims 20 to 23 wherein the patient is a CN patient.
